(19) United States
(12) Patent Application Publication
(10) Pub. No.: US 2011/0287008 A1
(43) Pub. Date: Nov. 24, 2011

(54) INHIBITION OF EMMPRIN TO TREAT MULTIPLE SCLEROSIS

(75) Inventors: Wee V. Yong, Calgary (CA); Smriti Agrawal, Calgary (CA); Claudia Silva, Calgary (CA)

(73) Assignee: UTI Limited Partnership, Calgary, AB (CA)

(21) Appl. No.: 12/994,361

(22) PCT Filed: May 21, 2009
(86) PCT No.: PCT/IB2009/006446

§ 371 Date: Aug. 3, 2011

Related U.S. Application Data
(60) Provisional application No. 61/055,760, filed on May 23, 2008.

(51) Int. Cl. A61K 39/395; A61K 31/7088; A61P 25/00

(52) U.S. Cl. 424/135.1; 424/158.1; 424/133.1; 514/44 A

(57) ABSTRACT

The present invention provides new methods for the prevention of migration of leukocytes into the CNS. These methods, which rely on inhibition of EMMPRIN activity or expression, can be used to treat or slow the onset of diseases such as multiple sclerosis and EAE.

The present application claims benefit of priority to U.S. Ser. No. 61/055,760, filed May 23, 2008, the entire contents of which are hereby incorporated by reference.

### I. FIELD OF THE INVENTION

The present invention relates to the fields of immunology, medicine and autoimmune disease. More particular, it addresses the use of inhibitor of EMMPRIN (CD 147) to prevent leukocyte infiltration into the CNS in inflammatory neurodegenerative diseases such as multiple sclerosis.

### II. BACKGROUND OF THE INVENTION

Multiple sclerosis (MS) is an immune-mediated disorder in which leukocytes cross the blood-brain barrier (BBB) to produce pathology within the CNS. Many of the pathological features of MS are reproduced in an animal model, experimental autoimmune encephalomyelitis (EAE). In both MS and EAE, there is a prerequisite of leukocytes traversing the BBB through a series of mechanisms that include the selective breakdown of a parenchymal basement membrane though the activity of matrix metalloproteinases (MMPs). There are 25 MMP members and many of these are implicated in the MS and EAE disease process (Yong et al., 2001). Moreover, several MMP members are simultaneously elevated in various compartments in MS and EAE, adding to the complexity of targeting MMP activity in these conditions. The genetic deletion of a single MMP member in mice has led invariably to the compensatory up-regulation of others, making it difficult to pinpoint particular MMP member for targeting. Moreover, existing pharmacological inhibitors of MMPs lack selectivity.

There remains a need for molecules and methods that effectively target MS and ameliorate its symptoms.

### SUMMARY OF THE INVENTION

The invention meets these needs and others by providing compositions and methods for the treatment of multiple sclerosis (MS). More specifically, the present invention relates to a method of inhibiting entry of leukocytes into the central nervous system (CNS) of a subject. The method comprises inhibiting entry of leukocytes into the central nervous system (CNS) of a subject by administering to the subject an effective amount of a molecule that inhibits the activity of EMMPRIN (CD147). The molecule may be an antibody or antibody fragment that binds immunologically to EMMPRIN, such as a scFv, scFab, Fab, chimeric or humanized antibody. The inhibiting activity may comprise inhibiting EMMPRIN expression, and the molecule may be an siRNA or miRNA that inhibits EMMPRIN expression. The siRNA may target an exon or an intron/exon junction. The miRNA may target an exon or an intron. The method may include use of two modalities, such as administering a least two distinct antibodies, such as one antibody that inhibits cell adhesion, such as leukocyte (e.g., monocyte) adhesion, and one antibody that inhibits cell proliferation, such as leukocyte (e.g., T cell) proliferation, at least two distinct siRNAs, or at least one antibody and one siRNA.

Administration may be by various routes, including intravenous, intraperitoneal, oral or via inhalation. The subject may suffer from multiple sclerosis (MS), and said molecule reduces one or more symptoms of multiple sclerosis (MS). The subject may suffer from multiple sclerosis (MS), and said molecule delays the progression of one or more symptoms of multiple sclerosis. The subject may be at risk of developing or has subclinical multiple sclerosis (MS), and said molecule delays the onset of one or more multiple sclerosis symptoms. The method may further comprise administering to said subject a second anti-MS therapy, such as Avonex®, CinnoVex®, ReciGen®, Rebif®, Betaseron®, Copaxone®, Novantrone®, or Tysabri®. The method may also further comprise at least a second administering of said molecule, such as is chronic administration.

The invention provides compositions such as reagents and formulations tailored to the subject methods.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F—EMMPRIN protein expression is upregulated in EAE. Immunofluorescence staining (FIGS. 1A-B), Western blot analysis (FIG. 1C) and flow cytometry (FIGS. 1D-F) revealed higher EMMPRIN protein expression in mice immunized for EAE, compared to wild-type controls. While EMMPRIN expression was restricted to blood vessels in control CNS (FIG. 1A, arrows), EMMPRIN was expressed by other cell types (arrow) as well as blood vessels in EAE (FIG. 1B). EMMPRIN protein levels, which were lowest in control (c) CNS, increased with EAE disease progression (days 5-15 post immunization), with the highest EMMPRIN expression at D15 (day 15) (FIG. 1C), when EAE disease was at its peak. In EAE mice (lower right FACS plot in FIG. 1D), the percentage of EMMPRIN-positive CD3+ cells was higher than wild-type controls. A time point analysis of EAE disease progression revealed increased EMMPRIN expression in T-cell (red bars) with EAE disease progression both in lymph nodes (FIG. 1E) and CNS (FIG. 1F). High macrophage (CD45+high CD11b+) expression of EMMPRIN (black bars) was detected in lymph nodes and spleen (FIG. 1E), and in the CNS (FIG. 1F) just before (D5) or at onset of clinical disease (D10). Microglia in the CNS (CD45+low, CD11b+) were high in EMMPRIN expression (FIG. 1F, green bars) only at disease onset, with EMMPRIN levels in these cells returning to normal at peak disease (D15).

FIGS. 2A-I—EMMPRIN expression on CNS resident and infiltrating cells in EAE. At peak EAE disease, various CNS cell types, both infiltrating CD45 positive cells (FIG. 2A) such as CD3+ T-cells (FIGS. 2B-C) and CNS resident cells such as GFAP-positive astrocytes (FIGS. 2D-G) and Iba-1-positive macrophage/microglia (FIGS. 2H-I) stained positive for EMMPRIN.

FIGS. 3A-D—High EMMPRIN levels detected in MS plaques. Both Western blots (FIGS. 3A-B) and immunofluorescence staining (FIGS. 3C-D) for EMMPRIN revealed high EMMPRIN levels in CNS samples from 5 different MS patients (MS1-5) versus healthy donors (C1 and C2). EMMPRIN protein levels were highest in the MS plaques (P) compared to adjacent white (W) and gray matter (G) from the CNS of the same patient (FIG. 3B). Using immunohistochemistry (FIG. 3C), it was found that EMMPRIN expression was on endothelial cells of blood vessels in a non-plaque containing area, while in an active lesion defined by hematoxylin-eosin and luxol fast blue (H&E/LFB), EMMPRIN was on cells resembling GFAP-positive astrocytes. Finally, double immunofluorescence analyses show that EMMPRIN in plaques are co-existent with GFAP, emphasizing that GFAP-positive astrocytes expressed high amounts of EMMPRIN in CNS from MS patients.

FIGS. 4A-H—Treatment with anti-EMMPRIN blocking antibody attenuates EAE disease severity. Mice immunized for EAE were treated with anti-EMMPRIN function blocking antibody or an isotype control at various time points post-immunization, and EAE disease onset and severity were documented (FIGS. 4A-C). EAE disease was attenuated in mice treated with anti-EMMPRIN antibody if injections encompassed days 8 and 11 post-immunization (FIGS. 4B-C), compared to groups treated with isotype control. Percentages of both CD45+/CD11b+macrophage/microglia (FIG. 4D) and CD45+/CD3+ T-cell (FIGS. 4E-G) in the CNS, were reduced in mice treated with anti-EMMPRIN compared to those treated with isotype control. Peripheral (lymph node) T-cell antigen response levels were comparable in both anti-EMMPRIN- and isotype control-treated groups (FIG. 4H). In all cases results are an average from at least 6 animals per group.

FIGS. 5A-B—Treatment with rEMMPRIN increases levels of proMMP-9 in murine NIH3T3 fibroblasts. Levels of proMMP-9 were measured by gelatin zymography or ELISA. 50 and 100K refers to the seeding density of cells, in thousands.

FIG. 6—Treatment with rEMMPRIN increases levels of MMP-9 in human monocytes. Monocytes were isolated from peripheral blood of normal adult volunteers through magnetic beads coated with an anti-CD14 antibody, so as to capture the CD14+ monocytes. Cells were then plated for 3 h in 20% serum-containing medium, at density of 100,000 or 200,000 (100 or 200K, respectively) per well. After switching medium over to serum-free medium, cells were incubated with 5 μg/ml recombinant human EMMPRIN (rEMMPRIN) for 24 h or 72 h, or were not treated (“nt”). The conditioned medium was then collected for zymography or ELISA. The results show that rEMMPRIN increases total MMP-9 at 24 or 72 h, with the latter time showing a more prominent increase.

FIG. 7—The level of MMP-9 expressed by activated human T cells is reduced by an anti-human EMMPRIN antibody. From peripheral blood of adult human volunteers, T cells were obtained by centrifugation with a Ficoll gradient. These cells were then activated with anti-CD3 (10 ng/mL), in the presence of anti-human EMMPRIN (UM-8D6, Ancell; 50 μg/ml) or an isotype antibody control (50 μg/ml). Conditioned media taken for MMP-9 analyses by gelatin zymography show that activated T cells in the presence of the anti-EMMPRIN antibody secrete less amounts of MMP-9 compared to cells in the presence of isotype antibody control

FIG. 8—Adhesion of monocytes onto fibronectin over a 30 min period was increased by an anti-human EMMPRIN antibody. Activation of monocytes by lipopolysaccharide (LPS, 100 ng/ml) increased binding onto fibronectin, but this level was surpassed by treatment of monocytes with anti-EMMPRIN (UM-8D6, Ancell; 10 μg/ml). Conversely, rEMMPRIN reduced binding. One implication of the results is that anti-EMMPRIN (with the RL73.2 anti-mouse EMMPRIN) treatment in the EAE model reduces leukocyte infiltration into the CNS parenchyma by preventing the production of MMP-9, and by increasing cell binding to the basement membrane thereby hindering the detachment of cells which is necessary for the next step of cellular locomotion.

FIG. 9—Anti-EMMPRIN antibody reduces the proliferation of human T cells activated by anti-CD3 plus anti-CD28. T cells were activated with 10 ng/ml anti-CD3 plus 10 ng/ml anti-CD28; anti-EMMPRIN (UM-8D6, Ancell) was used at 10 μg/ml. Mean±SD, n=4 wells/group. Proliferation measured at 3 days.

FIG. 10—Leukocytes are trapped in the perivascular space by anti-EMMPRIN treatment in EAE. Cerebellar brain slices are from mice induced for EAE and sacrificed 20 days after. Mice were treated with anti-mouse EMMPRIN (RL73.2 clone) at days 8, 11 and 15 after immunization with MOG or with isotype antibody control. The isotype antibody-treated animals had histological signs consistent with symptomatic EAE, where blood vessels are disrupted and where CD45+ leukocytes have dispersed into the CNS parenchyma. In contrast, anti-EMMPRIN treated mice had relatively well-preserved blood vessel structures, containing CD45+ leukocytes trapped within. Light regions are blood vessels stained with laminin; insets show concentration of CD45+ leukocytes in vessels. Red: CD45+ leukocytes; White: Blood vessels stained with a laminin antibody.

FIG. 11—When activated, T cells increase their levels of EMMPRIN. Murine CD4+ T cells were cultured in the absence (−) or presence (+) of anti-CD3 antibody which activates T cells. Conditioned media were then collected and used in Western blot analyses for EMMPRIN. A 55 kDa form of EMMPRIN was detected only in the conditioned medium collected from 2 different samples of activated T cells. Serum was used as a control.

FIGS. 12A-B—Structure of EMMPRIN and peptides used for antibody generation. (FIG. 12A) Structure of EMMPRIN, including amino acid residues flanking Asn44 glysosylation site at the ECI region. (FIG. 12B) Amino acid sequence at the ECI domain; those that differ between human and mouse are shown in red. The two peptide sequences selected for generation of monoclonal antibodies are displayed.

FIG. 13—Generation of antibodies to EMMPRIN. Different batches of polyclonal sera from mice raised to peptides 1 and 2 recognize rEMMPRIN and MS brain homogenates in a Western blot study. For each set, left to right: MS brain, rEMMPRIN, MW standard.

FIG. 14—Particular anti-EMMPRIN monoclonal antibodies reduce the proliferation of human T cells activated by anti-CD3. Act T: activated Y cells, whose proliferation rate should be taken as the activated control proliferation rate.

FIG. 15—Particular anti-EMMPRIN monoclonal antibodies reduce the number of human monocytes that adhere onto fibronectin. Mean±SEM, of 4 wells per group.

### DETAILED DESCRIPTION OF THE INVENTION

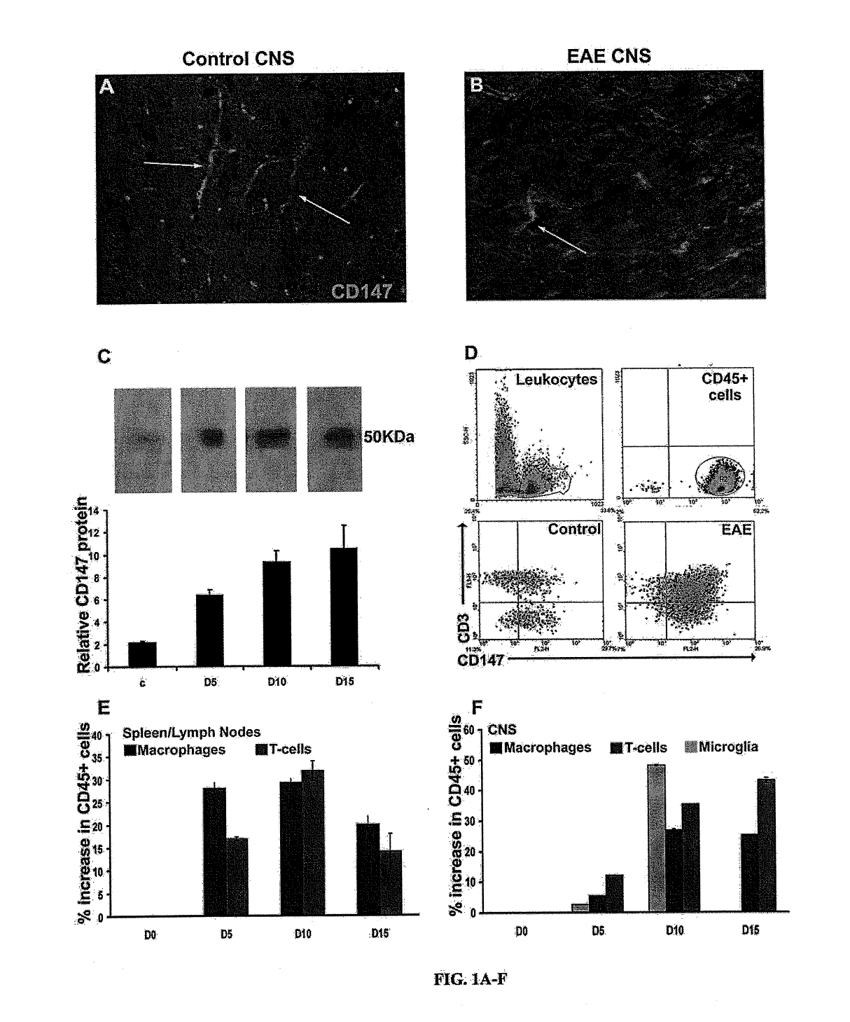

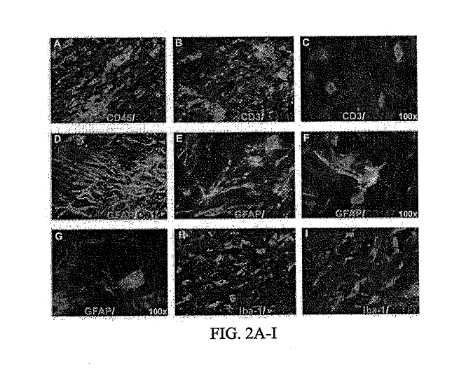

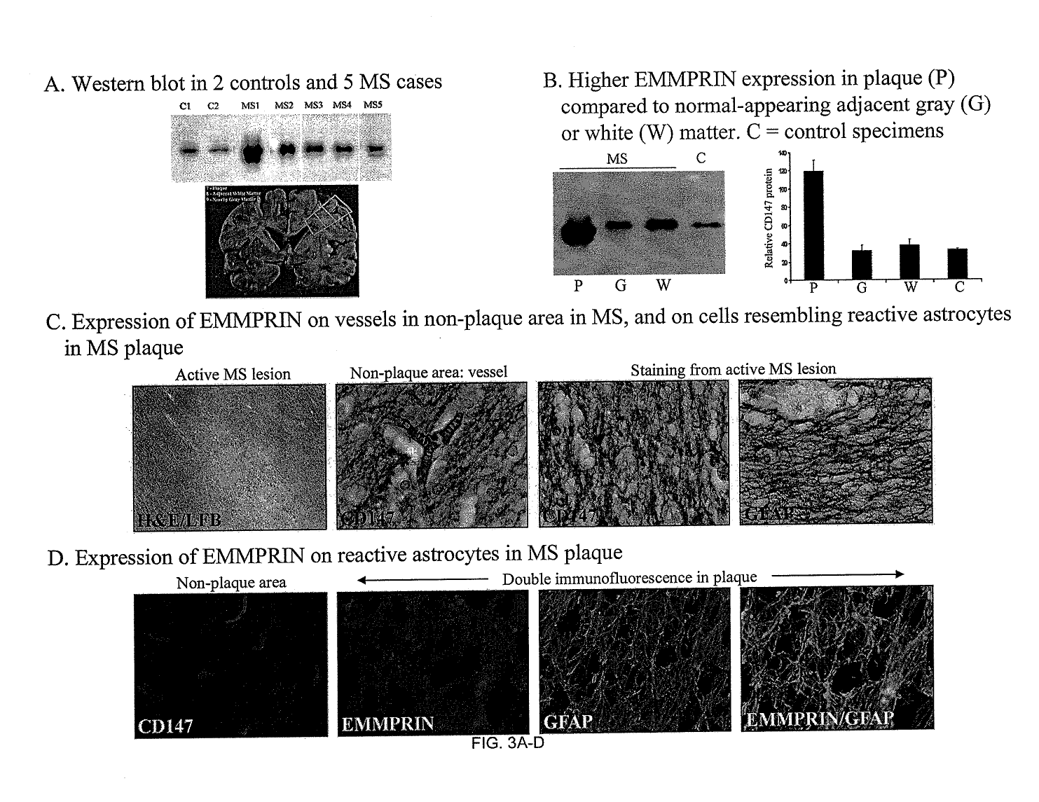

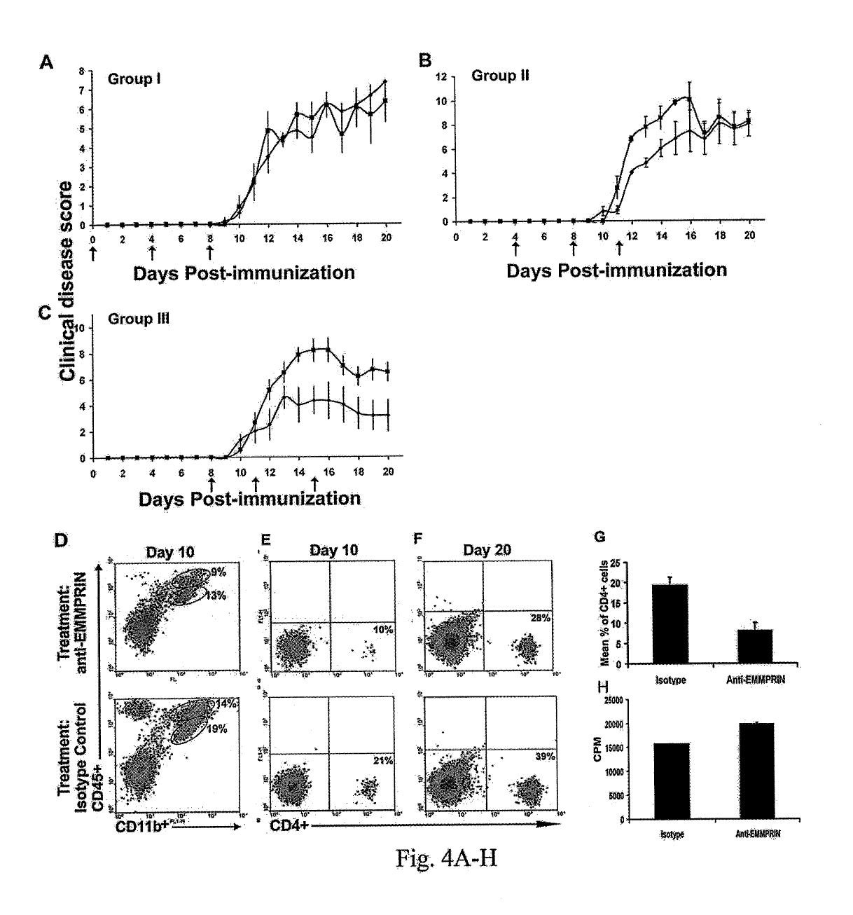

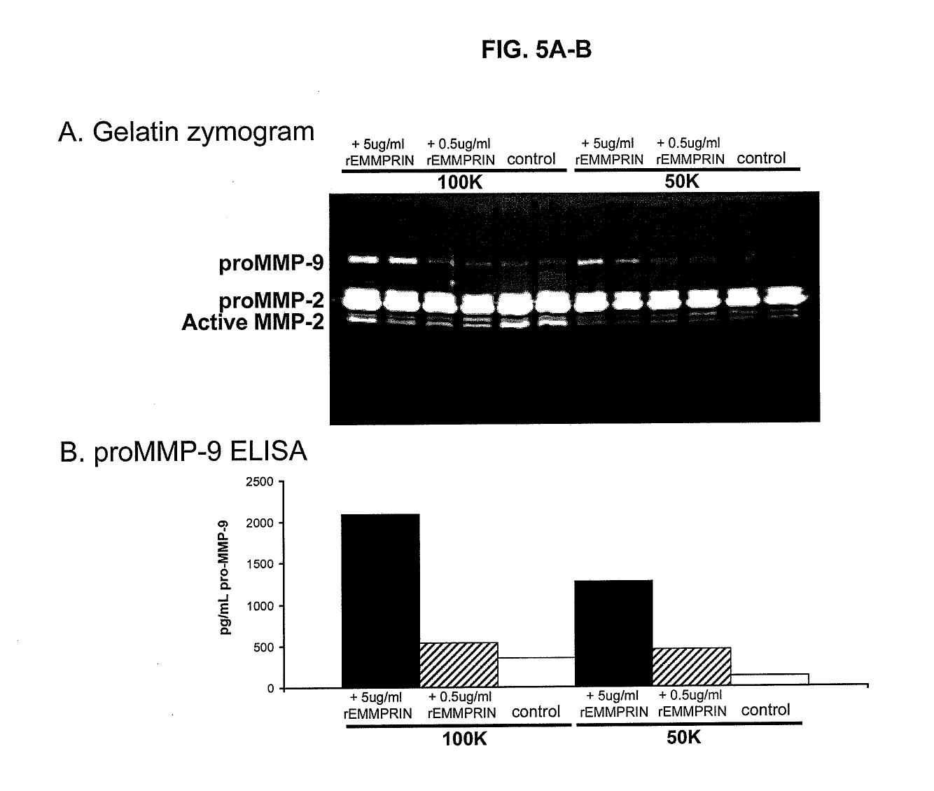

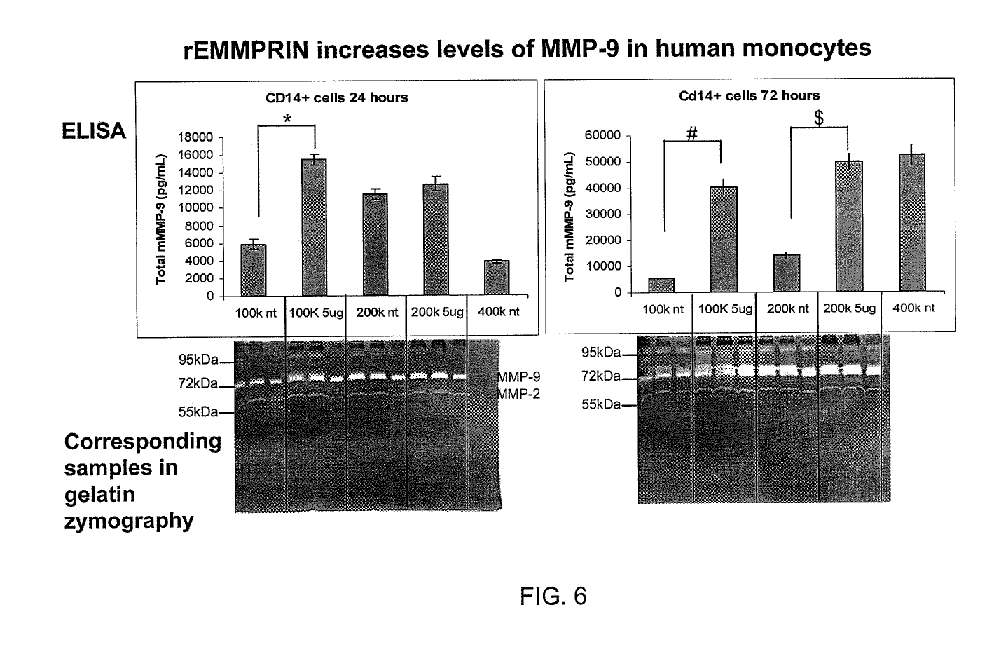

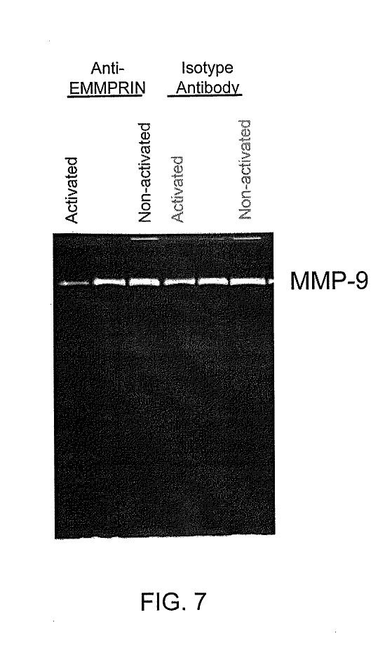

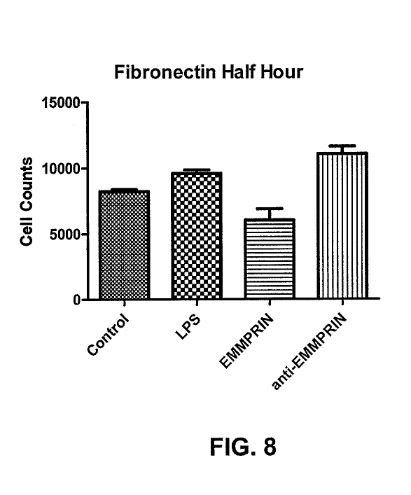

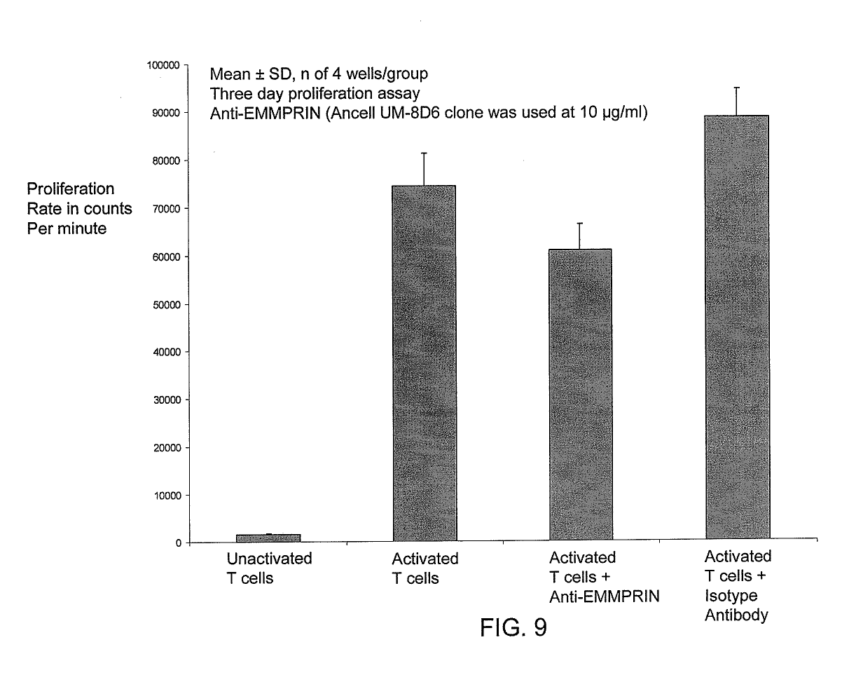

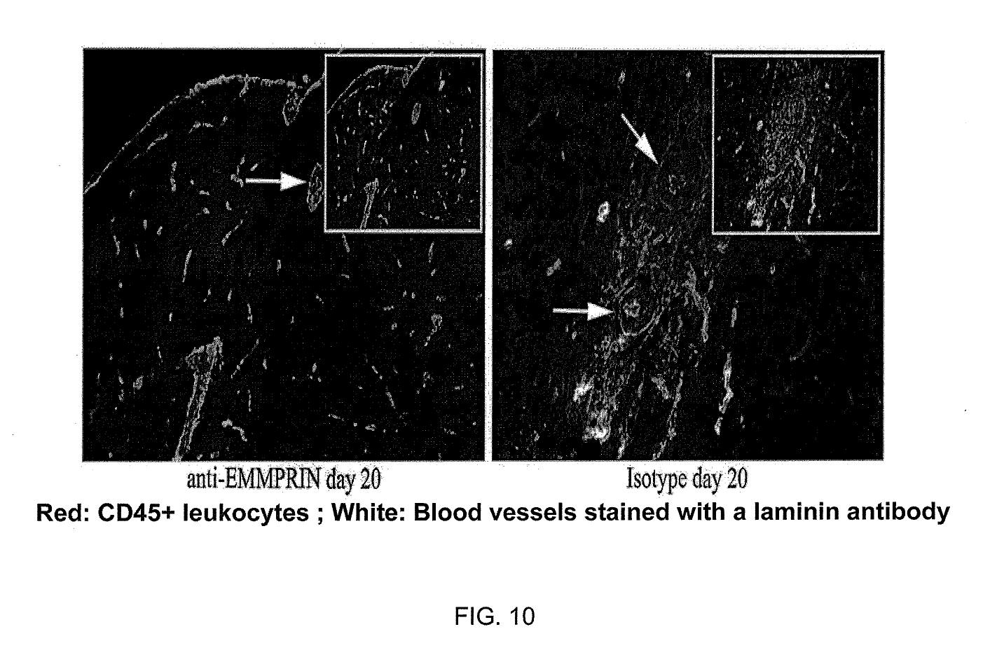

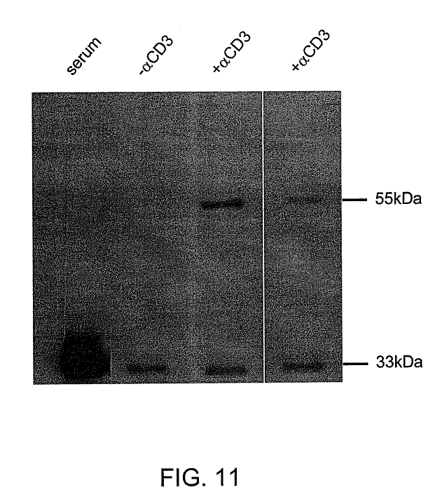

The present inventors are proposing a new approach to inhibit the entry of leukocytes into the CNS in MS, namely by inhibiting the extracellular matrix metalloproteinase inducer (EMMPRIN, also known as basigin or CD147) that is found on the surface of various cell types. EMMPRIN induces cells to produce and activate various MMPs, and can also regulate the activation status (e.g., proliferation) and adhesion properties of leukocytes; all of these characteristics influence the trafficking of leukocytes into the CNS, a prerequisite for disease activity in MS. The following findings indicate that inhibiting EMMPRIN is a potential MS therapy:
1. When T cells are activated in culture, they express high levels of glycosylated EMMPRIN. In EAE mice, the number of EMMPRIN-positive cells in the CNS increases from the onset of EAE signs and these continue to rise with disease progression (FIGS. 1A-C). EMMPRIN is detected in the CNS in both infiltrating (T-cells, macrophages) and resident (astrocytes/microglia) populations (FIGS. 1D and 2).
2. When cells are harvested temporally from the spleen and lymph nodes of EAE-affected mice and analyzed by flow cytometry, the increase of EMMPRIN in leukocytes is detected before the appearance of clinical signs (FIGS. 1E-F).
3. EMMPRIN levels are very high in brains obtained from patients with MS (FIG. 3A-D).
4. Mice treated with either an anti-EMMPRIN function blocking antibody (E-bioscience, clone RL73.2, 100 μg/mouse), or appropriate isotype control, injected at different time points of EAE disease show that anti-EMMPRIN antibody treatment results in a much lower EAE disease score (FIGS. 4A-C). By using flow cytometry analyses, the anti-EMMPRIN antibody treatment was found to reduce the number of CD4+ T cells, CD11b+CD45^{high} macrophages and CD11b+CD45^{low} microglia in the parenchyma of the CNS compared to animals treated with isotype controls (FIG. 4D-H). When tissue sections from EAE mice were analysed by immunohistochemistry, leukocytes detected by CD45 were found to be trapped within blood vessels in anti-EMMPRIN treated mice unlike the situation in mice treated with the isotype antibody control where leukocytes have dispersed into the CNS parenchyma (FIG. 10).
5. Tissue culture studies are also consistent with a pro-inflammatory effect of EMMPRIN. When mouse fibroblasts (FIG. 5A,B) or human monocytes (FIG. 6) were treated with recombinant EMMPRIN, they elevate their levels of MMP-9; conversely, anti-EMMPRIN treatment reduce levels of MMP-9 in activated T cells (FIG. 7).
6. When T cells are activated in culture with anti-CD3, they express high levels of glycosylated EMMPRIN (FIG. 11). Anti-EMMPRIN antibody treatment reduced the proliferation of T cells (FIG. 9).
7. Inhibiting EMMPRIN alters the adhesion of monocytes onto a fibronectin substrate (FIG. 8), a necessary step of leukocytes interacting with components of the blood-brain barrier before trafficking into the CNS parenchyma to produce disease.
   These findings suggest the importance of inhibiting EMMPRIN in MS to reduce the migration of leukocytes into the CNS to slow, prevent, alleviate and/or treat disease.

### I. DEFINITIONS

All scientific and technical terms used in this application have meanings commonly used in the art unless otherwise specified. As used in this application, the following words or phrases have the meanings specified.

As used herein, “symptoms of MS” include numbness or weakness in one or more limbs, loss of vision, pain during eye movement, double vision or blurring of vision, tingling or pain, electric-shock sensations that occur with certain head movements, tremor, lack of coordination or unsteady gait, fatigue, dizziness, an some cases, muscle stiffness or spasticity, slurred speech, paralysis, and problems with bladder or bowel control.

Amelioration of symptoms is typically determined by clinical evaluation. Those skilled in the art of neurological assessment are aware of appropriate tests and measures that can be used to give rise to a clinical score that indicates the extent of MS symptoms exhibited by an individual patient. Symptoms are considered ameliorated by a treatment when the clinical score after treatment is one-half or more units improved compared to the clinical score assessed prior to treatment. A typical assessment is known as the Multiple Sclerosis Functional Composite (MSFC) and is described, for example, in Ozakbas et al. (2004).

As used herein, “subject” means a mammal. Typically the mammal is a human. The subject can also be a veterinary subject, such as a canine, feline, rodent, equine, ovine, bovine subject.

The term “monoclonal antibody” (mAb) as used herein refers to an antibody obtained from a population of substantially homogeneous antibodies, i.e., the antibodies comprising the individual population are identical except for possible naturally-occurring mutations that may be present in minor amounts.

As used herein, “polypeptide” includes proteins, fragments of proteins, and peptides, whether isolated from natural sources, produced by recombinant techniques or chemically synthesized. Peptides of the invention typically comprise at least about 6 amino acids.

As used herein, “vector” means a construct, which is capable of delivering, and preferably expressing, one or more gene {s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various 15 types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18^{th} Ed., 1990).

### II. MULTIPLE SCLEROSIS

Multiple sclerosis (abbreviated MS, also known as disseminated sclerosis or encephalomyelitis disseminata) is an autoimmune condition in which the immune system attacks the central nervous system, leading to demyelination. Disease onset usually occurs in young adults, and it is more common in females. It has a prevalence that ranges between 2 and 150 per 100,000. MS was first described in 1868 by Jean-Martin Charcot. MS affects the ability of nerve cells in the brain and spinal cord to communicate with each other. Nerve cells communicate by sending electrical signals called action potentials down long fibers called axons, which are wrapped in an insulating substance called myelin. In MS, the body's own immune system attacks and damages the myelin. When myelin is lost, the axons can no longer effectively conduct signals. The name multiple sclerosis refers to scars (scleroses—better known as plaques or lesions) in the white matter of the brain and spinal cord, which is mainly composed of myelin. Although much is known about the mechanisms involved in the disease process, the cause remains unknown. Theories include genetics or infections. Different environmental risk factors have also been found.

Almost any neurological symptom can appear with the disease, and often progresses to physical and cognitive disability. MS takes several forms, with new symptoms occurring either in discrete attacks (relapsing forms) or slowly accumulating over time (progressive forms). Between attacks, symptoms may go away completely, but permanent neurological problems often occur, especially as the disease advances.

There is no known cure for MS. Treatments attempt to return function after an attack, prevent new attacks, and prevent disability (see detailed discussion below). MS medications can have adverse effects or be poorly tolerated, and many patients pursue alternative treatments, despite the lack of supporting scientific study. The prognosis is difficult to predict; it depends on the subtype of the disease, the individual patient's disease characteristics, the initial symptoms and the degree of disability the person experiences as time advances. Life expectancy of patients is nearly the same as that of the unaffected population.

Symptoms of MS usually appear in episodic acute periods of worsening (relapses, exacerbations, bouts or attacks), in a gradually-progressive deterioration of neurologic function, or in a combination of both.

The first clinical presentation of MS is referred to as clinically isolated syndrome (CIS). In CIS, a patient has an attack suggestive of demyelination, but does not fulfill the criteria for multiple sclerosis. Only 30 to 70% of persons experiencing CIS later develop MS. The disease usually presents with sensorial (46% of cases), visual (33%), cerebellar (30%) and motor (26%) symptoms. Many rare initial symptoms have also been reported, including aphasia, psychosis and epilepsy. Patients first seeking medical attention commonly present with multiple symptoms. The initial signs and symptoms of MS are often transient, mild, and self-limited. These signs and symptoms often do not prompt a person to seek medical attention and are sometimes identified only retrospectively once the diagnosis of MS has been made. Cases of MS are sometimes incidentally identified during neurological examinations performed for other causes. Such cases are referred to as subclinical MS.

The person with MS can suffer almost any neurological symptom or sign, including changes in sensation (hypoesthesia and paraesthesia), muscle weakness, muscle spasms, or difficulty in moving; difficulties with coordination and balance (ataxia); problems in speech (dysarthria) or swallowing (dysphagia), visual problems (nystagmus, optic neuritis, or diplopia), fatigue, acute or chronic pain, and bladder and bowel difficulties. Cognitive impairment of varying degrees and emotional symptoms of depression or unstable mood are also common. The main clinical measure of disability progression and symptom severity is the Expanded Disability Status Scale or EDSS.

Multiple sclerosis relapses are often unpredictable, occurring without warning and without obvious inciting factors. Some attacks, however, are preceded by common triggers. Relapses occur more frequently during spring and summer. Infections such as the common cold, influenza, or gastroenteritis increase the risk of relapse. Stress may also trigger an attack. Pregnancy may affect susceptibility to relapse, offering protection during the last trimester, for instance. During the first few months after delivery, however, the risk of relapse is increased. Overall, pregnancy does not seem to influence long-term disability. Many potential triggers have been examined and found not to influence MS relapse rates. There is no evidence that vaccination for influenza, hepatitis B, varicella, tetanus, or tuberculosis increases risk of relapse. Physical trauma does not trigger relapses. Exposure to higher than usual ambient temperatures can exacerbate extant symptoms, an effect known as Uhthoff's phenomenon. Uhthoff's phenomenon is not, however, an established relapse trigger.

Several subtypes, or patterns of progression, have been described. Subtypes use the past course of the disease in an attempt to predict the future course. They are important not only for prognosis but also for therapeutic decisions. In 1996 the United States National Multiple Sclerosis Society standardized four subtype definitions: relapsing remitting, secondary progressive, primary progressive and progressive relapsing.

The relapsing-remitting subtype is characterized by unpredictable relapses followed by periods of months to years of relative quiet (remission) with no new signs of disease activity. Deficits suffered during attacks may either resolve or leave sequelae. This describes the initial course of 85-90% of individuals with MS. When deficits always resolve between attacks, this is sometimes referred to as benign MS.

Secondary progressive MS describes those with initial relapsing-remitting MS, who then begin to have progressive neurologic decline between acute attacks without any definite periods of remission. Occasional relapses and minor remissions may appear. The median time between disease onset and conversion from relapsing-remitting to secondary progressive MS is 19 years.

The primary progressive subtype describes the approximately 10-15% of individuals who never have remission after their initial MS symptoms. It is characterized by progression of disability from onset, with no, or only occasional and minor, remissions and improvements. The age of onset for the primary progressive subtype is later than other subtypes.

Progressive relapsing MS describes those individuals who, from onset, have a steady neurologic decline but also suffer clear superimposed attacks. This is the least common of all subtypes.

Cases with non-standard behavior have also been described. Sometimes referred to as borderline forms of multiple sclerosis, these include Devic's disease, Balo concentric sclerosis, Schilder's diffuse sclerosis and Marburg multiple sclerosis. Multiple sclerosis also behaves differently in children. There is debate whether these are atypical variants of MS or different diseases.

Multiple sclerosis can be difficult to diagnose since its signs and symptoms may be similar to many other medical problems. Medical organizations have created diagnostic criteria to ease and standardize the diagnostic process for practicing physicians. Historically, the Schumacher and Poser criteria were both popular. Currently, the McDonald criteria focus on a demonstration with clinical, laboratory and radiologic data of the dissemination of MS lesions in time and space. A diagnosis cannot be made until other possible conditions have been ruled out and there is evidence of demyelinating events separated anatomically and in time.

Clinical data alone may be sufficient for a diagnosis of MS if an individual has suffered separate episodes of neurologic symptoms characteristic of MS. Since some people seek medical attention after only one attack, other testing may hasten and ease the diagnosis. The most commonly used diagnostic tools are neuroimaging, analysis of cerebrospinal fluid and evoked potentials. Magnetic resonance imaging of the brain and spine shows areas of demyelination (lesions or plaques). Gadolinium can be administered intravenously as a contrast to highlight active plaques and, by elimination, demonstrate the existence of historical lesions not associated with symptoms at the moment of the evaluation. Testing of cerebrospinal fluid obtained from a lumbar puncture can provide evidence of chronic inflammation of the central nervous system. The cerebrospinal fluid is tested for oligoclonal bands, which are an inflammation marker found in 75-85% of people with MS. The nervous system of a person with MS often responds less actively to stimulation of the optic nerve and sensory nerves due to demyelination of such pathways. These brain responses can be examined using visual and sensory evoked potentials.

MS is currently believed to be an immune-mediated disorder with an initial trigger, which may have a viral etiology, although this concept has been debated for years and some still oppose it. Damage is believed to be caused by the patient's own immune system. The immune system attacks the nervous system, possibly as a result of exposure to a molecule with a similar structure to one of its own.

MS lesions most commonly involve white matter areas close to the ventricles of the cerebellum, brain stem, basal ganglia and spinal cord; and the optic nerve. The function of white matter cells is to carry signals between grey matter areas, where the processing is done, and the rest of the body. The peripheral nervous system is rarely involved.

More specifically, MS destroys oligodendrocytes, the cells responsible for creating and maintaining a fatty layer—known as the myelin sheath—which helps the neurons carry electrical signals. MS results in a thinning or complete loss of myelin and, as the disease advances, the cutting (transection) of the neuron's extensions or axons. When the myelin is lost, a neuron can no longer effectively conduct electrical signals. A repair process, called remyelination, takes place in early phases of the disease, but the oligodendrocytes cannot completely rebuild the cell's myelin sheath. Repeated attacks lead to successively fewer effective remyelinations, until a scar-like plaque is built up around the damaged axons. Four different lesion patterns have been described.

Apart from demyelination, the other pathologic hallmark of the disease is inflammation. According to a strictly immunological explanation of MS, the inflammatory process is caused by T cells, a kind of lymphocyte. Lymphocytes are cells that play an important role in the body's defenses. In MS, T cells gain entry into the brain via the blood-brain barrier, a capillary system that should prevent entrance of T cells into the nervous system. The blood-brain barrier is normally not permeable to these types of cells, unless triggered by infection or a virus, which decreases the integrity of the tight junctions forming the barrier. When the blood-brain barrier regains its integrity, usually after infection or virus has cleared, the T cells are trapped inside the brain. The T cells recognize myelin as foreign and attack it as if it were an invading virus. This triggers inflammatory processes, stimulating other immune cells and soluble factors like cytokines and antibodies. Leaks form in the blood-brain barrier, which in turn cause a number of other damaging effects such as swelling, activation of macrophages, and more activation of cytokines and other destructive proteins.

Experimental autoimmune encephalomyelitis, sometimes Experimental Allergic Encephalomyelitis (EAE) is an animal model of brain inflammation. It is an inflammatory demyelinating disease of the central nervous system (CNS). It is mostly used with rodents and is widely studied as an animal model of the human CNS demyelinating diseases, including the diseases multiple sclerosis and acute disseminated encephalomyelitis (ADEM). EAE was motivated by observations during the convalescence from viral diseases by Rivers and co-workers in 1933.

EAE can be induced by inoculation with whole CNS tissue, purified myelin basic protein (MBP) or myelin proteolipid protein (PLP), together with adjuvants. It may also be induced by the passive transfer of T cells specifically reactive to these myelin antigens. EAE may have either an acute or a chronic relapsing course. Acute EAE closely resembles the human disease acute disseminated encephalomyelitis, while chronic relapsing EAE resembles multiple sclerosis. EAE is also the prototype for T-cell-mediated autoimmune disease in general.

### III. ANTIBODIES

The term antibody as used in the present application refers to single anti-EMMPRIN monoclonal antibodies (including antagonist and neutralizing antibodies) and anti-EMMPRIN antibody compositions with polyepitopic specificity. Such antibodies bind to EMMPRIN proteins and polypeptides. Specific antibodies will specifically bind to an EMMPRIN protein and will not bind (or will bind weakly) to non-EMMPRIN proteins and polypeptides. Anti-EMMPRIN antibodies that are particularly contemplated include monoclonal and polyclonal antibodies as well as fragments containing the antigen binding domain and/or one or more complementarity determining regions of these antibodies. As used herein, an antibody fragment is defined as at least a portion of the variable region of the immunoglobulin molecule that binds to its target, i.e., the antigen binding region.

Particular antibodies and antibody fragments of the invention specifically bind to the cell surface, extracellular portion of the CD147/EMMPRIN protein. The antigen sequence for RL73.2, one of the clones used to produce anti-CD147 for studies described herein, can be found in Renno et al. (2002). This antibody targeting mouse EMMPRIN is commercially available from eBioscience. Another anti-EMMPRIN used for studies described herein is the UM-8D6 clone, which is targeted towards human EMMPRIN and is commercially available from Ancell.

Useful in therapeutic methods for treatment of multiple sclerosis are systemically administered EMMPRIN antibodies that interfere with EMMPRIN function. Inhibiting EMMPRIN function includes but is not limited to one or more of the following:
(1) inhibiting EMMPRIN induction of MMP levels and/or activity: glycosylation and homophilic interaction of EMMPRIN (see Urn et al., 1998; Toole, 2003); intracellular signaling through the MAP-kinase p38 pathway and arachidonate metabolism (see Taylor et al., 2002); caveolin-1 binding-mediated reversion of EMMPRIN from an MMP antagonist to agonist;
(2) inhibiting EMMPRIN cleavage by MMP-14, resulting in active 22 kDa secreted form (see Egawa et al., 2006);
(3) inhibiting EMMPRIN binding to cyclophillin (Gwinn et al., 2006);
(4) inhibiting EMMPRIN expression (Curtin et al., 2007); and
(5) inhibiting EMMPRIN binding to monocarboxylate transporter (MCT-1) on astrocytes (Korn et al., 2005).
   The inhibited activity also may be assessed from the standpoint of the affect on EMMPRIN target cells, for example, such as inhibiting cell adhesion, such as monocyte adhesion, and inhibiting cell proliferation, such as T cell proliferation. Various methods for the preparation of antibodies are well known in the art. For example, antibodies may be prepared by immunizing a suitable mammalian host using an EMMPRIN protein, peptide, or fragment, in isolated or immunoconjugated form (Harlow and Lane, 1988; Harlow, 1989). In addition, fusion proteins of EMMPRIN may also be used, such as an EMMPRIN GST-fusion protein. In another embodiment, an EMMPRIN peptide may be synthesized and used as an immunogen.

The antibodies or fragments may also be produced, using current technology, by recombinant means. Regions that bind specifically to the desired regions of the EMMPRIN protein can also be produced in the context of chimeric or CDR-grafted antibodies of multiple species origin. Humanized or human EMMPRIN antibodies may also be produced and are preferred for use in therapeutic contexts. Methods for humanizing murine and other non-human antibodies by substituting one or more of the non-human antibody CDRs for corresponding human antibody sequences are well known (see for example, Jones et al., 1986; Riechmann et al., 1988; Verhoeyen et al., 1988). See also, Carter et al. (1993), and Sims et al. (1993). Methods for producing fully human monoclonal antibodies include phage display and transgenic methods (for review, see Vaughan et al., 1998).

Fully human EMMPRIN monoclonal antibodies may be generated using cloning technologies employing large human Ig gene combinatorial libraries (i.e., phage display) (Griffiths and Hoogenboom, 1993; Burton and Barbas, 1992). Fully human EMMPRIN monoclonal antibodies may also be produced using transgenic mice engineered to contain human immunoglobulin gene loci as described in PCT Patent Application WO98/24893; Jakobovits (1998). This method avoids the in vitro manipulation required with phage display technology and efficiently produces high affinity authentic human antibodies.

Reactivity of EMMPRIN antibodies with an EMMPRIN protein may be established by a number of well known means, including western blot, immunoprecipitation, ELISA, and FACS analyses using, as appropriate, EMMPRIN proteins, peptides, EMMPRIN-expressing cells or extracts thereof. An EMMPRIN antibody that can block EMMPRIN function can be determined using a tissue culture bioassay in which activated T cells interact with astrocytes to lead to the upregulation of various matrix metalloproteinase members, including MMP-9. The ability of anti-EMMPRIN antibodies to block this MMP-9 upregulation in T cell-astrocyte interaction can be assessed. An antibody is considered to disrupt the biological activity of EMMPRIN (CD147) if it blocks this MMP9 upregulation. Alternately, the ability of anti-EMMPRIN antibodies to block the proliferation of anti-CD3 activated T cells (for example, using the assay described in FIG. 9), or to increase or decrease the adhesion of monocytes onto a fibronectin substrate (for example, using the assay described in FIG. 8), could be examined.

### IV. INHIBITORY NUCLEIC ACID MOLECULES

### A. Antisense Molecules

The antisense molecules of the present invention comprise a sequence substantially complementary, or preferably fully complementary, to all or a fragment of an EMMPRIN gene. Included are fragments of oligonucleotides within the coding sequence of an EMMPRIN gene, and inhibitory nucleotides that inhibit the expression of EMMPRIN. EMMPRIN specific siRNA are available from Qiagen, (Valencia, Calif.; GenBank Accession No. NM_{—}009768 and NM_{—}001077184). Antisense oligonucleotides of DNA or RNA complementary to sequences at the boundary between introns and exons can be employed to prevent the maturation of newly-generated nuclear RNA transcripts of specific genes into mRNA for transcription. Antisense RNA, including siRNA, complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

Antisense compositions of the invention include oligonucleotides formed of homopyrimidines that can recognize local stretches of homopurines in the DNA double helix and bind to them in the major groove to form a triple helix. See: Helen and Toulme (1990). Formation of the triple helix would interrupt the ability of the specific gene to undergo transcription by RNA polymerase. Triple helix formation using myc-specific oligonucleotides has been observed. See: Cooney et al., (1988).

Antisense RNA can be provided to the cell as “ready-to-use” RNA synthesized in vitro or as an antisense gene stably transfected into cells which will yield antisense RNA upon transcription. Hybridization with mRNA results in degradation of the hybridized molecule by RNAse H and/or inhibition of the formation of translation complexes. Both result in a failure to produce the product of the original gene.

### B. RNAi

RNA interference (also referred to as “RNA-mediated interference” or RNAi) is another mechanism by which protein expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy)thymidine nucleotides as the di-nucleotide overhangs. These di-nucleotide overhangs are often written as dTdT to distinguish them from the typical nucleotides incorporated into RNA. The literature has indicated that the use of dTdT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. See U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25-mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

### C. Synthesis

Both antisense RNA and DNA, siRNA molecules and ribozymes of the invention may be prepared by any method known in the art for the synthesis of RNA molecules. These include techniques for chemically synthesizing oligonucleotides such as solid phase phosphoramidite chemical synthesis. Alternatively, RNA molecules may be generated by in vitro or in vivo transcription of DNA sequences encoding the antisense RNA molecule. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

### D. Modifications

A wide variety of well-known, alternative oligonucleotide chemistries may be used (see, e.g., U.S. Patent Publications 2007/0213292, 2008/0032945, 2007/0287831, etc.), particularly single-stranded complementary oligonucleotides comprising 2′ methoxyethyl, 2′-fluoro, and morpholino bases (see e.g., Summerton and Weller, 1997). The oligonucleotide may include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). Also contemplated are locked nucleic acid (LNA) and peptide nucleic acids (PNA).

Peptide nucleic acids (PNAs) are nonionic DNA mimics that have outstanding potential for recognizing duplex DNA (Kaihatsu et al., 2004; Nielsen et al., 1991). PNAs can be readily synthesized and bind to complementary sequences by standard Watson-Crick base-pairing (Egholm et al., 1993), allowing them to target any sequence within the genome without the need for complex synthetic protocols or design considerations. Strand invasion of duplex DNA by PNAs is not hindered by phosphate-phosphate repulsion and is both rapid and stable (Kaihatsu et al., 2004; Nielsen et al., 1991). Applications for strand invasion by PNAs include creation of artificial primosomes (Demidov et al., 2001), inhibition of transcription (Larsen and Nielsen, 1996), activation of transcription (Mollegaard et al., 1994), and directed mutagenesis (Faruqi et al., 1998). PNAs would provide a general and potent strategy for probing the structure and function of chromosomal DNA in living systems if their remarkable strand invasion abilities could be efficiently applied inside cells.

Strand invasion by PNAs in cell-free systems is most potent at sequences that are partially single-stranded (Bentin and Nielsen, 1996; Zhang et al., 2000). Assembly of RNA polymerase and transcription factors into the pre-initiation complex on DNA induces the formation of a structure known as the open complex that contains several bases of single-stranded DNA (Holstege et al., 1997; Kahl et al., 2000). The exceptional ability of PNAs to recognize duplex DNA allows them to intercept the open complex of an actively transcribed gene without a requirement for preincubation. The open complex is formed during transcription of all genes and PNAs can be synthesized to target any transcription initiation site. Therefore, antigene PNAs that target an open complex at a promoter region within chromosomal DNA would have the potential to be general tools for controlling transcription initiation inside cells.

A locked nucleic acid (LNA), often referred to as inaccessible RNA, is a modified RNA nucleotide (Elmén et al., 2008). The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. Such oligomers are commercially available. The locked ribose conformation enhances base stacking and backbone pre-organization. This significantly increases the thermal stability (melting temperature) of oligonucleotides (Kaur et al., 2006). LNA bases may be included in a DNA backbone, by they can also be in a backbone of LNA, 2′-O-methyl RNA, 2′-methoxyethyl RNA, or 2′-fluoro RNA. These molecules may utilize either a phosphodiester or phosphorothioate backbone.

Other oligonucleotide modifications can be made to produce oligonucleotides. For example, stability against nuclease degradation has been achieved by introducing a phosphorothioate (P═S) backbone linkage at the 3′ end for exonuclease resistance and 2′ modifications (2′-OMe, 2′-F and related) for endonuclease resistance (WO 2005115481; Choung et al., 2006). A motif having entirely of 2′-O-methyl and 2′-fluoro nucleotides has shown enhanced plasma stability and increased in vitro potency (Allerson et al., 2005). The incorporation of 2′-O-Me and 2′-O-MOE does not have a notable effect on activity (Prakash et al., 2005).

Sequences containing a 4′-thioribose modification have been shown to have a stability 600 times greater than that of natural RNA (Hoshika et al, 2004). Crystal structure studies reveal that 4′-thioriboses adopt conformations very similar to the C3′-endo pucker observed for unmodified sugars in the native duplex (Haeberli et al., 2005). Stretches of 4′-thio-RNA were well tolerated in both the guide and non-guide strands. However, optimization of both the number and the placement of 4′-thioribonucleosides is necessary for maximal potency.

In the boranophosphate linkage, a non-bridging phosphodiester oxygen is replaced by an isoelectronic borane (BH3-) moiety. Boranophosphate siRNAs have been synthesized by enzymatic routes using T7 RNA polymerase and a boranophosphate ribonucleoside triphosphate in the transcription reaction. Boranophosphate siRNAs are more active than native siRNAs if the center of the guide strand is not modified, and they may be at least ten times more nuclease resistant than unmodified siRNAs (Hall et al., 2004; Hall et al., 2006).

Certain terminal conjugates have been reported to improve or direct cellular uptake. For example, NAAs conjugated with cholesterol improve in vitro and in vivo cell permeation in liver cells (Rand et al., 2005). Soutschek et al. (2004) have reported on the use of chemically-stabilized and cholesterol-conjugated siRNAs have markedly improved pharmacological properties in vitro and in vivo. Chemically-stabilized siRNAs with partial phosphorothioate backbone and 2′-O-methyl sugar modifications on the sense and antisense strands (discussed above) showed significantly enhanced resistance towards degradation by exo- and endonucleases in serum and in tissue homogenates, and the conjugation of cholesterol to the 3′ end of the sense strand of an oligonucleotides by means of a pyrrolidine linker does not result in a significant loss of gene-silencing activity in cell culture. These study demonstrates that cholesterol conjugation significantly improves in vivo pharmacological properties of oligonucleotides.

U.S. Patent Publication 2008/0015162, incorporated herein by reference, provide additional examples of nucleic acid analogs useful in the present invention. The following excerpts are derived from that document and are exemplary in nature only.

In certain embodiments, oligomeric compounds comprise one or more modified monomers, including 2′-modified sugars, such as BNA's and monomers (e.g., nucleosides and nucleotides) with 2′-substituents such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂₋₀—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

In certain embodiments, the oligomeric compounds including, but no limited to short oligomers of the present invention, comprise one or more high affinity monomers provided that the oligomeric compound does not comprise a nucleotide comprising a 2′—O(CH₂)ₙH, wherein n is one to six. In certain embodiments, the oligomeric compounds including, but no limited to short oligomers of the present invention, comprise one or more high affinity monomer provided that the oligomeric compound does not comprise a nucleotide comprising a 2′-OCH₃ or a 2′-O(CH₂)₂OCH₃. In certain embodiments, the oligomeric compounds comprise one or more high affinity monomers provided that the oligomeric compound does not comprise a α-L-methyleneoxy (4′-CH₂—O-2′) BNA and/or a β-D-methyleneoxy (4′-CH₂—O-2′) BNA.

Certain BNA's have been prepared and disclosed in the patent literature as well as in scientific literature (Singh et al., 1998; Koshkin et al., 1998; Wahlestedt et al., 2000; Kumar et al., 1998; WO 94/14226; WO 2005/021570; Singh et al, 1998; examples of issued U.S. patents and published applications that disclose BNA s include, for example, U.S. Pat. Nos. 7,053,207; 6,268,490; 6,770,748; 6,794,499; 7,034,133; and 6,525,191; and U.S. Patent Publication Nos. 2004/0171570; 2004/0219565; 2004/0014959; 2003/0207841; 2004/0143114; and 2003/0082807.

Also provided herein are BNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., 2001; Braasch et al., 2001; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) BNA is used for the bicyclic moiety; in the case of an ethylene group in this position, the term ethyleneoxy (4′-CH₂CH₂—O-2′) BNA is used (Singh et al., 1998; Morita et al., 2003). Methyleneoxy (4′-CH₂—O-2′) BNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., 2000).

An isomer of methyleneoxy (4′-CH₂—O-2′) BNA that has also been discussed is α-L-methyleneoxy (4′-CH₂—O-2′) BNA which has been shown to have superior stability against a 3′-exonuclease. The α-L-methyleneoxy (4′-CH₂—O-2′) BNA's were incorporated into antisense gapmers and chimeras that showed potent antisense activity (Frieden et al., 2003).

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., 1998). BNAs and preparation thereof are also described in WO 98/39352 and WO 99/14226.

Analogs of methyleneoxy (4′-CH₂—O-2′) BNA, phosphorothioate-methyleneoxy (4′-CH₂—O-2′) BNA and 2′-thio-BNAs, have also been prepared (Kumar et al., 1998). Preparation of Locked Nucleoside Analogs Comprising Oligodeoxyribonucleotide Duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., 1998). In addition, 2′-amino- and 2′-methylamino-BNA's have been prepared and the thermal stability of their duplexes with complementary RNA and DNA strands has been previously reported.

Modified sugar moieties are well known and can be used to alter, typically increase, the affinity of oligomers for targets and/or increase nuclease resistance. A representative list of modified sugars includes, but is not limited to, bicyclic modified sugars (BNA's), including methyleneoxy (4′-CH₂—O-2′) BNA and ethyleneoxy (4′-(CH₂)₂—O-2′ bridge) BNA; substituted sugars, especially 2′-substituted sugars having a 2′-F, 2′-OCH₃ or a 2′—O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

### E. Delivery

A variety of methods may be used to deliver oligonucleotides into a target cell. For cells in vitro embodiments, delivery can often be accomplished by direct injection into cells, and delivery can often be enhanced using hydrophobic or cationic carriers. Alternatively, the cells can be permeabilized with a permeabilization and then contacted with the oligonucleotide. The oligomer can be administered to the subject either as a naked oligonucleotide agent, in conjunction with a delivery reagent, or as a recombinant plasmid or viral vector which expresses the oligonucleotide agent.

For cells in situ, several applicable delivery methods are well-established, e.g., Elmen et al. (2008), Akinc et al. (2008); Esau et al. (2006), Krützfeldt et al. (2005). In particular, cationic lipids (see e.g., Hassani et al., 2005) and polymers such as polyethylenimine (see e.g., Urban-Klein, 2005) have been used to facilitate oligonucleotide delivery. Compositions consisting essentially of the oligomer (i.e., the oligomer in a carrier solution without any other active ingredients) can be directly injected into the host (see e.g., Tyler et al., 1999; McMahon et al., 2002). In vivo applications of duplex RNAs are reviewed in Paroo and Corey (2004).

When microinjection is not an option, delivery can be enhanced in some cases by using Lipofectamine™ (Invitrogen, Carlsbad, Calif.). PNA oligomers can be introduced into cells in vitro by complexing them with partially complementary DNA oligonucleotides and cationic lipid. The lipid promotes internalization of the DNA, while the PNA enters as cargo and is subsequently released. Peptides such as penetratin, transportan, Tat peptide, nuclear localization signal (NLS), and others, can be attached to the oligomer to promote cellular uptake (see e.g., Kaihatsu et al., 2003; Kaihatsu et al., 2004). Alternatively, the cells can be permeabilized with a permeabilization agent such as lysolecithin, and then contacted with the oligomer.

Alternatively, certain oligonucleotide agents featured in the instant invention can be expressed within cells from eukaryotic promoters (e.g., Izant and Weintraub, 1985; McGarry and Lindquist, 1986; Scanlon et al., 1991; Kashani-Sabet et al., 1992; Weerasinghe et al., 1991; Ojwang et al., 1992; Chen et al., 1992; Sarver et al., 1990; Thompson et al., 1995). Those skilled in the art realize that any nucleic acid can be expressed in eukaryotic cells from the appropriate DNA/RNA vector. The activity of such nucleic acids can be augmented by their release from the primary transcript by a enzymatic nucleic acid (PCT WO 93/23569; PCT WO 94/02595; Ohkawa et al., 1992; Taira et al., 1991; Ventura et al., 1993; Chowrira et al., 1994).

The recombinant vectors can be DNA plasmids or viral vectors. Oligonucleotide agent-expressing viral vectors can be constructed based on, but not limited to, adeno-associated virus, retrovirus, adenovirus, or alphavirus. In another embodiment, pol III based constructs are used to express nucleic acid molecules of the invention (see for example Morris et al., 2004; U.S. Pat. Nos. 5,902,880 and 6,146,886). The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the oligomer interacts with the target sequence. In a particular embodiment, the oligomer forms a duplex with target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (see Couture et al., 1996).

Methods for the delivery of nucleic acid molecules are also described in Agrawal and Akhtar (1995), Akhtar et al. (1992), Maurer et al. (1999), all of which are incorporated herein by reference. U.S. Pat. No. 6,395,713 and PCT WO 94/02595 and WO 00/53722 further describe general methods for delivery of nucleic acid molecules.

Oligonucleotide compositions oligonucleotides, derivatives and analogs thereof, conjugation protocols, and strategies for inhibition of transcription and translation are generally described in Antisense Research and Applications, Nielsen, 1993; Nucleic Acids in Chemistry and Biology, 1990; and Oligonucleotides and Analogues: A Practical Approach, 1991; which are each hereby incorporated herein by reference including all references cited therein which are hereby incorporated herein by reference.

### V. PHARMACEUTICAL COMPOSITIONS

The invention provides polypeptides or polynucleotides that are incorporated into pharmaceutical compositions. Pharmaceutical compositions comprise one or more such compounds and, optionally, a physiologically acceptable carrier.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous, intradermal or intramuscular administration. For parenteral administration, such as subcutaneous injection, the carrier preferably comprises water, saline, alcohol, a fat, a wax or a buffer. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption or penetration across the blood-brain barrier of the delivered molecule. Such excipients are those substances usually and customarily employed to formulate dosages for parenteral administration in either unit dose or multi-dose form or for direct infusion into the CSF by continuous or periodic infusion from an implanted pump.

Such compositions may also comprise buffers (e.g., neutral buffered saline or phosphate buffered saline), carbohydrates (e.g., glucose, mannose, sucrose or dextrans), mannitol, proteins, peptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site, such as a site of surgical excision of a tumor. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

### VI. THERAPEUTIC AND PROPHYLACTIC METHODS

Treatment includes prophylaxis and therapy. Prophylaxis or therapy can be accomplished by a single direct administration of an EMMPRIN inhibitory treatment at a single time point at a single or multiple sites, or at multiple time points to a single or multiple sites. Administration can also be nearly simultaneous to multiple sites. Patients or subjects include mammals, such as human, bovine, equine, canine, feline, porcine, and ovine animals. The subject is, in particular, a human, including one having a disease state associated with leukocyte infiltration into the CNS, such as MS.

The EMMPRIN inhibitory treatment may also comprises a combination of inhibitors, such as two antibodies with similar or distinct activities, two distinct siRNA's, or a combination of an anti-EMMPRIN antibody and an EMMPRIN siRNA.

### A. Administration and Dosage

The EMMPRIN inhibitor compositions are administered in any suitable manner, often with pharmaceutically acceptable carriers, although more than one route can be used to administer a particular composition, and particular route can provide a more immediate and more effective response than another route.

The dose administered to a patient, in the context of the present invention, should be sufficient to effect a beneficial therapeutic response in the patient over any period of time, or to inhibit disease progression. Thus, the composition is administered to a subject in an amount sufficient to alleviate, reduce, cure or at least partially arrest symptoms and/or complications from the disease. An amount adequate to accomplish any of these is defined as a “therapeutically effective dose.”

Routes and frequency of administration of the therapeutic compositions disclosed herein, as well as dosage, will vary from individual to individual, and may be readily established using standard techniques. In general, the pharmaceutical compositions may be administered, by injection (e.g., intracutaneous, intramuscular, intravenous or subcutaneous), intranasally (e.g., by aspiration) or orally. More specifically, between 1 and 10 doses may be administered over a 52 week period. Particularly, 6 doses are administered, at intervals of 1 month, and additional administrations may be given periodically thereafter. Alternate protocols may be appropriate for individual patients. In one embodiment, two intravenous injections of the composition are administered 10 days apart.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### B. Kits

For use in the therapeutic applications described herein, kits are also within the scope of the invention. Such kits can comprise a carrier, package or container that is compartmentalized to receive one or more containers such as vials, tubes, and the like, each of the container(s) comprising one of the separate elements to be used in the method. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic application, and can also indicate directions for either in vivo or in vitro use, such as those described above. Directions and or other information can also be included on an insert which is included with the kit.

### C. Combination Therapies

The use of combination treatments is a common therapeutic approach. This can have the benefit of enhancing therapeutic efficacy of the combined agents, and/or reducing the amount of drug needed to achieve the same benefit as compared to either drug alone, while simultaneously reducing side effects therefrom. Such combinations may involve an anti-EMMPRIN treatment that precedes, is co-current with and/or follows the other therapy by intervals ranging from minutes to weeks. In embodiments where the anti-EMMPRIN treatment and other agent(s) are applied separately to a cell, tissue or organism, one would generally ensure that a significant period of time did not expire between the time of each delivery, such that the anti-EMMPRIN treatment and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the anti-EMMPRIN treatment. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the anti-EMMPRIN treatment.

Various combination regimens of the anti-EMMPRIN treatment and one or more agents may be employed. Non-limiting examples of such combinations are shown below, wherein an anti-EMMPRIN treatment is “A” and an agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

What follows is a discussion of various MS therapies than can be used with the anti-EMMPRIN treatments discussed above.

Although there is no known cure for multiple sclerosis, several therapies have proven helpful. The primary aims of therapy are returning function after an attack, preventing new attacks, and preventing disability. As with any medical treatment, medications used in the management of MS have several adverse effects. Alternative treatments are pursued by some patients, despite the shortage of supporting, comparable, replicated scientific study.

During symptomatic attacks, administration of high doses of intravenous corticosteroids, such as methylprednisolone, is the routine therapy for acute relapses. The aim of this kind of treatment is to end the attack sooner and leave fewer lasting deficits in the patient. Although generally effective in the short term for relieving symptoms, corticosteroid treatments do not appear to have a significant impact on long-term recovery. Potential side effects include osteoporosis and impaired memory, the latter being reversible.

Disease-modifying treatments are expensive and most of these require frequent (up-to-daily) injections. Others require IV infusions at 1-3 month intervals. The earliest clinical presentation of relapsing-remitting MS (RRMS) is the clinically isolated syndrome (CIS). Several studies have shown that treatment with interferons or glatiramer acetate after an initial attack can decrease the chance that a patient will develop clinical MS.

As of 2007, six disease-modifying treatments have been approved by regulatory agencies of different countries for RRMS. Three are interferons: two formulations of interferon β1a (tradenames Avonex, CinnoVex, ReciGen and Rebif) and one of interferon β1b (U.S. tradename Betaseron, in Europe and Japan Betaferon). A fourth medication is glatiramer acetate (Copaxone). The fifth medication, mitoxantrone, is an immunosuppressant also used in cancer chemotherapy, approved only in the USA and largely for secondary progressive MS. The sixth is natalizumab (marketed as Tysabri). All six medications are modestly effective at decreasing the number of attacks and slowing progression to disability, although their efficacy rates differ, and studies of their long-term effects are still lacking. Comparisons between immunomodulators (all but mitoxantrone) show that the most effective is natalizumab, both in terms of relapse rate reduction and halting disability progression; it has also been shown to reduce the severity of MS. Mitoxantrone may be the most effective of them all; however, it is generally not considered as a long-term therapy, as its use is limited by severe cardiotoxicity.

The interferons and glatiramer acetate are delivered by frequent injections, varying from once-per-day for glatiramer acetate to once-per-week (but intra-muscular) for Avonex. Natalizumab and mitoxantrone are given by IV infusion at monthly intervals. Immunomodulators are in Phase III trials for MS, and also are contemplated in accordance with the present invention, including cladribine, laquinimod, minocycline, fingolimod, terfluonomide, fumarate, alemtuzumab and rituximab.

Treatment of progressive MS is more difficult than relapsing-remitting MS. Mitoxantrone has shown positive effects in patients with secondary progressive and progressive relapsing courses. It is moderately effective in reducing the progression of the disease and the frequency of relapses in patients in short-term follow-up. No treatment has been proven to modify the course of primary progressive MS.

As with any medical treatment, these treatments have several adverse effects. One of the most common is irritation at the injection site for glatiramer acetate and the interferon treatments. Over time, a visible dent at the injection site, due to the local destruction of fat tissue, known as lipoatrophy, may develop. Interferons produce symptoms similar to influenza; some patients taking glatiramer experience a post-injection reaction manifested by flushing, chest tightness, heart palpitations, breathlessness, and anxiety, which usually lasts less than thirty minutes. More dangerous are liver damage from interferons and mitoxantrone, the immunosuppressive effects and cardiac toxicity of the latter; and the putative link between natalizumab and some cases of progressive multifocal leukoencephalopathy.

Disease-modifying treatments reduce the progression rate of the disease, but do not stop it. As multiple sclerosis progresses, the symptomatology tends to increase. The disease is associated with a variety of symptoms and functional deficits that result in a range of progressive impairments and disability. Management of these deficits is therefore very important. Both drug therapy and neurorehabilitation have shown to ease the burden of some symptoms, though neither influences disease progression. As for any patient with neurologic deficits, a multidisciplinary approach is key to limiting and overcoming disability; however, there are particular difficulties in specifying a ‘core team’ because people with MS may need help from almost any health profession or service at some point. Similarly, for each symptom there are different treatment options. Treatments should therefore be individualized depending both on the patient and the physician.

As with most chronic diseases, alternative treatments are pursued by some patients, despite the shortage of supporting, comparable, replicated scientific study. Examples are dietary regimens, herbal medicine, including the use of medical cannabis to help alleviate symptoms, and hyperbaric oxygenation. The therapeutic practice of martial arts such as tai chi, relaxation disciplines such as yoga, or general exercise seems to mitigate fatigue, but has no effect on cognitive function.

### D. Industrial Applicability

The invention described herein provides a “migration inhibitor” to selectively prevent the entry of leukocytes into the CNS without non-specifically or extensively reducing T cell proliferation. Targeting EMMPRIN with a monoclonal antibody allows one to create a highly specific therapeutic that will inhibit the production and activation of MMPs at the blood-brain barrier (BBB) when leukocytes are seeking entry into the CNS. Some medications in use for MS do influence MMPs, an unsurprising finding given the importance of MMPs to the disease process. Specifically, the interferons can reduce the production of some MMPs by leukocytes, while the experimental therapy minocycline can inhibit MMP enzymatic activity but with low potency. However, none of the currently approved MS therapies target MMPs specifically (or effectively). The approach of this invention not only targets a broad spectrum of MMPs, it would be most effective at the crucial stage when MMPs are produced in large quantities—during the active disease stage when cell-cell (or extracellular matrix protein) contact regulates the migration of leukocytes into the CNS, e.g., between leukocyte-astrocyte/microglia, or between leukocyte-fibronectin and other extracellular matrix proteins at the basement membranes of the blood-brain barrier. This allows one to address the underlying MS disease process rather than trying to dampen the generalized immune response during periods of active MS.

### VII. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Targeting EMMPRIN-Dependent Early Metalloproteinase Induction in Murine Experimental Autoimmune Encephalomyelitis (EAE)

Matrix metalloproteinases (MMPs) have been shown to have detrimental roles in neuroinflammatory diseases such as multiple sclerosis (MS). However, the fact that MMP family members interact with, compensate for, and may even be inhibited by each other has made the biology of MMPs in MS a difficult one to unravel. Several MMP members are simultaneously elevated in MS and in EAE, a model of MS, and this has added to the complexity of targeting singular or multiple MMP members in MS/EAE.

A new approach to affect MMPs in MS could be to alter the mechanisms or molecules that regulate the early expression and activity of MMPs in disease states. The extracellular matrix metalloproteinase inducer (EMMPRIN, also called basigin or CD147) is found on the surface of various cell types in an inactive state. When highly glycosylated leading to its activation, EMMPRIN induces fibroblasts and tumor cells to produce and activate various MMPs.

The inventors show that the activation of antigen (MOG35-55) specific T-cells in culture leads to their secretion of high levels of active EMMPRIN. In vivo, and by utilizing both immunohistology and FACS analysis, increased numbers of EMMPRIN-positive cells are found in the CNS of mice from the onset of EAE signs, compared to controls, and these continued to rise with disease progression. EMMPRIN immunoreactivity is detected in the CNS in both infiltrating (T-cells, macrophages) and resident (astrocytes and microglia, not neurons) cellular populations. When cells are harvested temporally from the spleen and lymph nodes and analyzed by FACS, the increase of EMMPRIN in leukocytes is detected before the appearance of clinical signs.

### Example 2

### Alleviation of Pathology via Inhibition of EMMPRIN Activity

The above results suggest that EMMPRIN plays a critical role in activating MMPs in leukocytes and CNS cells; EMMPRIN may have other as yet unknown roles in leukocytes and CNS cells associated with neuroinflammation. To test whether inhibiting EMMPRIN activity would alleviate neuroinflammation and pathology, the inventors investigated mice treated with either an anti-EMMPRIN function blocking antibody (E-bioscience, clone RL73, 100 μg/mouse), or appropriate isotype control, at different time points of EAE disease. The inventors found that anti-EMMPRIN antibody treatment results in a much lower EAE disease score and fewer CD4+ T cells as well as CD45+ macrophages/microglia in the CNS compared to animals treated with isotype controls (FIGS. 4A-E). These findings suggest the importance of inhibiting EMMPRIN in MS to reduce leukocyte migration into the CNS to alleviate disease.

### Example 3

### Optimization of Time Course for Treatment

This example illustrates, using the EAE mouse model of MS, determination of the best time frame and dosage to inhibit EMMPRIN during EAE. The following study was conducted to evaluate time points of treatment. The same protocol can be readily adapted for evaluation of dosage and other parameters.

Mice immunized for EAE were treated with anti-EMMPRIN function blocking antibody or an isotype control at various time points post-immunization with a disease inducing myelin peptide, MOG35-55, and EAE disease onset and severity were documented (FIGS. 4A-C). EAE disease was only attenuated in mice treated with anti-EMMPRIN antibody given at least twice at days 8 and 11 post immunization (FIGS. 4B-C), compared to groups treated with isotype control. Percentages of both CD45+/CD11b+ macrophage/microglia (FIG. 4D) and CD45+/CD3+ T-cell (FIGS. 4E-G) in the CNS, were reduced in mice treated with anti-EMMPRIN compared to those treated with isotype control. T-cell antigen response levels were comparable in both anti-EMMPRIN and isotype control treated groups (FIG. 4H). In all cases results are an average from at least 6 animals per group.

CNS tissues can be collected at onset and other time points of EAE disease to determine differences in MMP levels (via gelatin and in situ zymography), alteration in cytokine levels, as well as altered cellular responses (utilizing flow cytometry), in 7 isotype versus anti-EMMPRIN treated mice, as well as in comparisons of differing doses and other treatment parameters.

### Example 4

### Further Defining the Role of EMMPRIN in EAE

As shown in FIGS. 5A-B and FIG. 6, recombinant EMMPRIN increases levels of MMP-9 in murine fibroblasts and human monocytes. This is important since MMP-9 is used by leukocytes to traffic into the CNS and therefore inhibiting EMMPRIN (to prevent leukocyte trafficking) is important.

Another commercial anti-human EMMPRIN antibody (UM-8D6, Ancell) showed changes in the activation status of the immune cells. Specifically, the level of MMP-9 expressed by activated human T cells is reduced by an anti-human EMMPRIN antibody (FIG. 7). This could therefore suggest reduced trafficking of leukocytes into the CNS by treatment with an anti-human EMMPRIN antibody in MS.

### Example 5

### Additional Evidence that Inhibition of EMMPRIN can Block EAE

The same anti-EMMPRIN antibody shown in FIG. 7 reduces the proliferation of human T cells (T cells that were activated by anti-CD3 and anti-CD28) (FIG. 9). This implies that the anti-EMMPRIN antibody may be able to reduce leukocyte activity in MS.

Anti-EMMPRIN antibody (Ancell antibody) also increases the adhesiveness of monocytes (on a fibronectin substrate in culture) (FIG. 8), which supports the hypothesis that the use of an anti-EMMPRIN antibody may help trap leukocytes in the blood brain barrier and prevent them from entering the CNS.

Finally, histology from the previously used anti-EMMPRIN antibody (RL73.2-eBioscience)-treated EAE mice reveals that the leukocytes appear to be trapped in blood vessels, supporting the idea that anti-EMMPRIN treatment prevents migration of leukocytes into the CNS parenchyma and thus reduces disease manifestation (FIG. 10).

### Example 6

### Monoclonal Antibodies to EMMPRIN

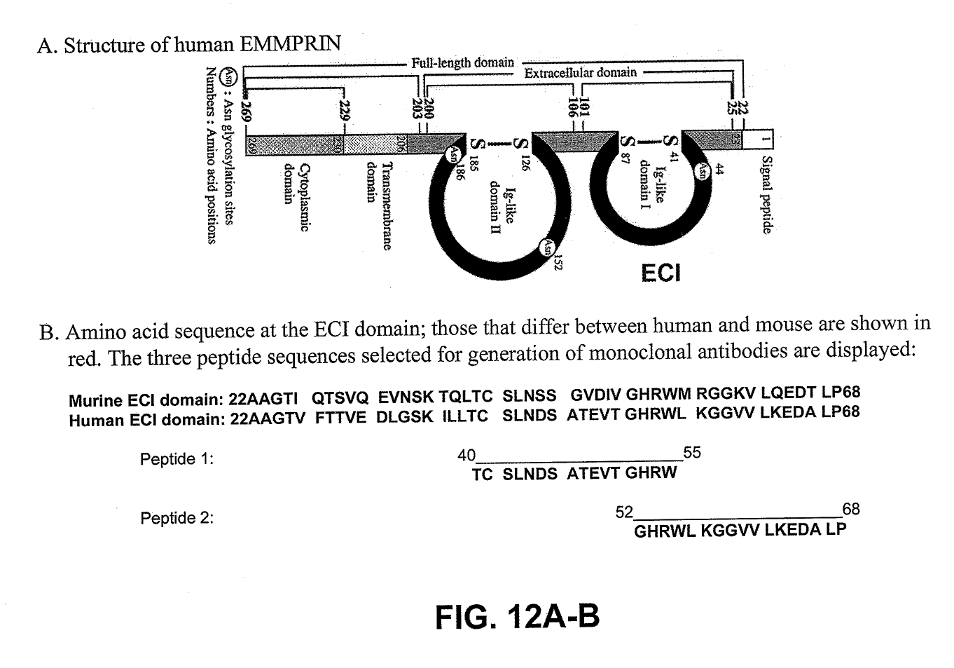

The inventors synthesized two peptides (peptide 1 and 2; FIG. 12B) that are based on amino acid sequences at the ECI domain of human EMMPRIN. Peptide 1 of 16 amino acids spans amino acid sequence 40 to 55, while peptide 2 of 17 amino acids spans amino acid sequence 52 to 68 at the ECI domain of human EMMPRIN (FIG. 12A). These two human sequence peptides have some amino acid identity to the mouse sequence (FIG. 12B); thus, besides being able to target human EMMPRIN, it is possible that the antibodies generated may also recognize mouse EMMPRIN, which would be useful in future EAE mouse experiments.

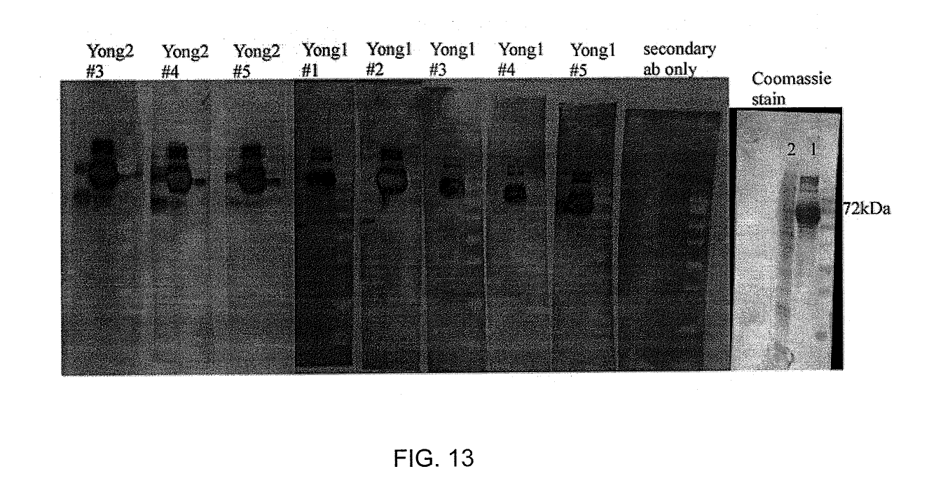

Given that the polyclonal sera from mice immunized with peptides 1 and 2 recognize EMMPRIN (FIG. 13), hybridoma clones that produce monoclonal antibodies to EMMPRIN have been generated. The inventors tested these monoclonal antibodies (henceforth labeled Yong1mab#) for their ability to reduce the proliferation of human T cells that are activated by anti-CD3, or to perturb the adhesion of human monocytes onto fibronectin.

To assess whether the Yong1mab hybridoma clones could affect the proliferation of activated T cells, blood was taken from normal human volunteers and mononuclear cells were obtained by Ficoll centrifugation. Cells were then plated at 200 k/well of 96 well plates and T cells were activated using 10 ng/ml anti-CD3 and 10 ng/ml anti CD28. Cells were treated with an antibody to EMMPRIN (Ancell clone UM-8D6) or isotype antibody as positive and negative controls, respectively. In other wells, conditioned media from “Yong1mab” hybridoma clones producing monoclonal antibodies to EMMPRIN were added to the T cells. Conditioned media were added 1:1 with T cell culture medium. Cells were left for 72 h whereby 1 μCi/well of tritium (³H-thymdiine) was added at the 48 h mark. Cells were harvested and the amount of radioactivity incorporated into cellular DNA was analysed by liquid scintillation counting that provided counts per minute (cpm).

Next, the inventors sought to assess whether the Yong1mab hybridoma clones could affect the adhesion of human monocytes onto fibronectin. The adhesion of leukocytes onto the parenchymal basement membrane of the blood-brain barrier is an important step in the subsequent trafficking of leukocytes into the parenchyma of the central nervous system (Agrawal et al., 2006). The basement membrane is comprised of extracellular matrix molecules such as fibronectin. Thus, the adhesion of leukocytes onto fibronectin has been used as an indicator of migratory capacity into the parenhcyma. In this regard, too weak a binding affinity to fibronectin (fewer cells attach) reflects the inability to adhere and thus to further migrate, while too strong a binding affinity can also be counterproductive for migration by virtue of cells being stuck on the matrix.

Human monocytes were isolated from peripheral blood of human volunteers by magnetic beads coated with anti-CD14 antibodies (from Miltenyi Biotec). These were then plated onto wells of 96-well plates previously coated with fibronectin at 10 μg/ml for 4 h at 37° C. Seeding density was 50,000 cells per well. When anti-EMMPRIN monoclonal antibodies were used, the conditioned medium collected from hybridoma cells were incubated 1:1 with monocytes contained within monocyte culture medium, and the whole mixture (50,000 cells in 100 μl medium) was then added onto individual wells of 96-well plates coated with fibronectin.

After 30 min, the number of cells adhered to fibronectin was determined. In this regard, the medium was gently aspirated from wells, a gentle PBS wash was done, and 4% paraformaldehyde was then applied. Cells were then stained with CD14-PE to label specifically monocytes, and with Hoescht dye to label all nuclei. The number of cells adhered per well was obtained from the sum of 4 fields sampled at precise areas around the center of a well by ImageXpress automated counting (Molecular Dynamics).

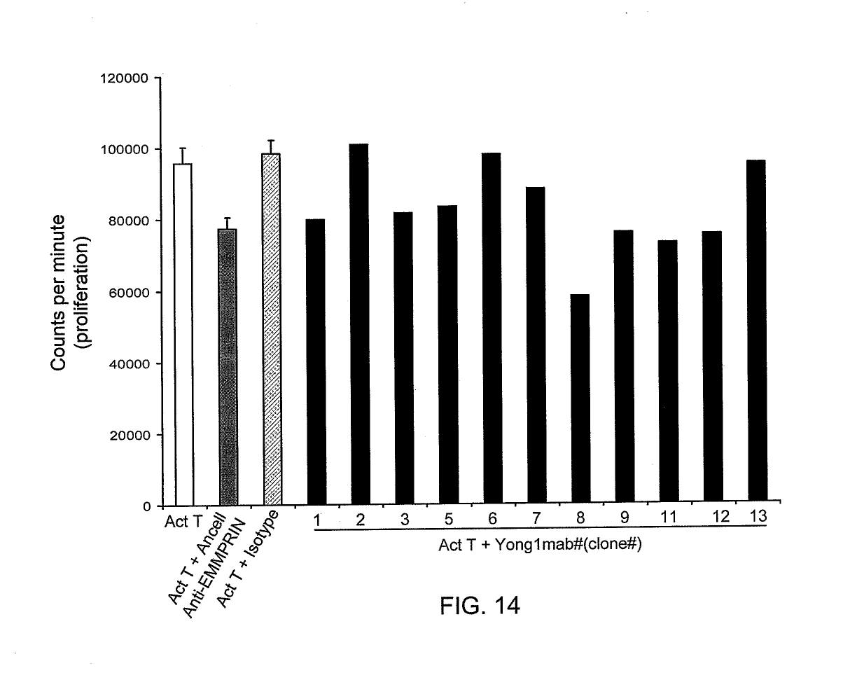

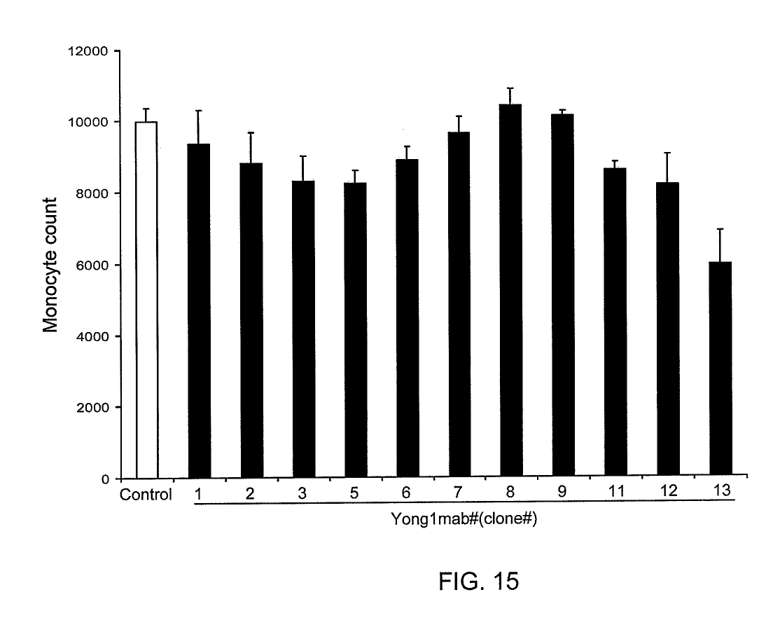

Particular monoclonal antibodies have inhibitory effects on either T cell proliferation (Yong1mab clones #1, 3, 5, 8, 9, 11, 12) (FIG. 14) or monocyte adhesion (Yong1mab clones #3, 5, 11, 12, 13) (FIG. 15), or both (Yong1mab clones #3, 5, 11, 12). The inventors will determine in the future clones that inhibit MMP-9 levels, or those that detect human and mouse EMMPRIN in Western blots, or those that inhibit the manifestation of EAE.

The foregoing description and examples are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### VIII. REFERENCES

The following references, to the extent that they provide exemplary procedural or other details supplementary to those set forth herein, are specifically incorporated herein by reference:
U.S. Pat. No. 4,415,732
U.S. Pat. No. 4,458,066
U.S. Pat. No. 4,897,268
U.S. Pat. No. 4,981,957
U.S. Pat. No. 5,075,109
U.S. Pat. No. 5,118,800
U.S. Pat. No. 5,319,080
U.S. Pat. No. 5,359,044
U.S. Pat. No. 5,393,878
U.S. Pat. No. 5,446,137
U.S. Pat. No. 5,466,786
U.S. Pat. No. 5,514,785
U.S. Pat. No. 5,519,134
U.S. Pat. No. 5,567,811
U.S. Pat. No. 5,576,427
U.S. Pat. No. 5,591,722
U.S. Pat. No. 5,597,909
U.S. Pat. No. 5,610,300
U.S. Pat. No. 5,627,053
U.S. Pat. No. 5,639,873
U.S. Pat. No. 5,646,265
U.S. Pat. No. 5,658,873
U.S. Pat. No. 5,670,633
U.S. Pat. No. 5,700,920
U.S. Pat. No. 5,792,747
U.S. Pat. No. 5,795,715
U.S. Pat. No. 5,889,136
U.S. Pat. No. 5,902,880
U.S. Pat. No. 6,146,886
U.S. Pat. No. 6,268,490
U.S. Pat. No. 6,395,713
U.S. Pat. No. 6,525,191
U.S. Pat. No. 6,531,584
U.S. Pat. No. 6,600,032
U.S. Pat. No. 6,670,461
U.S. Pat. No. 6,770,748
U.S. Pat. No. 6,794,499
U.S. Pat. No. 7,034,133
U.S. Pat. No. 7,053,207
U.S. Patent Publn. 2003/0082807
U.S. Patent Publn. 2003/0207841
U.S. Patent Publn. 2004/0014959
U.S. Patent Publn. 2004/0143114
U.S. Patent Publn. 2004/0171570
U.S. Patent Publn. 2004/0219565
U.S. Patent Publn. 2007/0213292
U.S. Patent Publn. 2007/0287831
U.S. Patent Publn. 2008/0015162
U.S. Patent Publn. 2008/0032945
Agrawal and Akhtar, Trends Biotechnol., 13(6):197-9, 1995.
Agrawal et al., J. Exp. Med., 203:1007, 2006.
Akhtar et al., Trends Cell Bio., 2:139, 1992.
Akinc et al. Nat. Biotechnol., 26(5):561-9, 2008.
Allerson et al., J. Med. Chem., 48:901-904, 2005.
Bentin and Nielsen, Biochemistry, 35:8863-8869, 1996.
Bosher and Labouesse, Nat. Cell. Biol., 2(2):E31-E36, 2000.
Braasch et al., Chem. Biol., 8(1):1-7, 2001.
Burton and Barbas, In: Human Antibodies from combinatorial libraries. Academic Titles, Nottingham, 65-82, 1992.
Caplen et al., Gene, 252(1-2):95-105, 2000.
Carter et al., Proc. Natl. Acad. Sci. USA, 89:4285, 1993.
Chen et al., Fed. of Eur. Biochem Soc., 309:115-118, 1992.
Choung et al., Biochem. Biophys. Res. Commun., 342:919-927, 2006.
Chowrira et al., J. Biol. Chem., 269(41):25856-25864, 1994.
Cooney et al., Science, 241(4864):456-459, 1988.
Couture et al., Proc. Natl. Acad. Sci. USA, 93:754-758, 1996.
Curtin et al., Glia, 55(15):1542-53 2007.
Demidov et al., ChemBiochem., 2:133-139, 2001.
Egawa et al., J. Biol. Chem., 281(49):37576-37585, 2006.
Egholm et al., Nature, 365(6446):566-568, 1993.
Elayadi et al., Nucleic Acids Res., 29(8):1683-9, 2001.
Elbashir et al., EMBO, 20(23):6877-6888, 2001.
Elbashir et al., Genes Dev., 5(2):188-200, 2001.
Elbashir et al., Nature, 411(6836):494-498, 2001.
Elmén et al., Nature, 452(7189):896-9, 2008.
Esau et al., Cell Metab., 3(2):87-98, 2006.
Faruqi et al., Proc. Natl. Acad. Sci. USA, 95:1398-1403, 1998.
Fire et al., Nature, 391(6669):806-811, 1998.
Frieden et al., Nucl. Nucleic Acids, 22:1041-1043, 2003.
Griffiths and Hoogenboom, In: Protein Engineering of Antibody Molecules for Prophylactic and Therapeutic Applications in Man, Clark (Ed.), Nottingham Academic, 45-64, 1993.
Grishok et al., Science, 287:2494-2497, 2000.
Gwinn et al., J. Immunol., 177:4870-4879, 2006.
Haeberli et al., Nucleic Acids Res., 33:3965-3975, 2005.
Hall et al., Nucleic Acids Res., 32:5991-6000, 2004.
Hall et al., Nucleic Acids Res., 34:2773-2781, 2006.
Harlow and Lane, Antibodies: A Laboratory Manual, CSH Press, Eds., 1988.
Harlow, Antibodies, Cold Spring Harbor Press, NY, 1989.
Hassani et al., J. Gene Med., 7(2):198-207, 2005.
Helen and Toulme, Biochem. Biophys Acta, 1049:99-125, 1990.
Holstege et al., EMBO J., 16:7468-7480, 1997.
Hoshika et al., Nucleic Acids Res., 32:3815-3825, 2004.
Izant and Weintraub, Science, 229(4711):345-52, 1985.
Jakobovits, Exp. Opin. Invest. Drugs, 7(4):607-614, 1998.
Jones et al., Nature, 321: 522-525, 1986.
Kahl et al., J. Mol. Biol., 299:75-89, 2000.
Kaihatsu et al., Biochemistry, 42(47):13996-4003, 2003.
Kaihatsu et al., Biochemistry, 43(45):14340-7, 2004.
Kaihatsu et al., Chem. Biol., 11:749-758, 2004.
Kashani-Sabet et al., Antisense Res. Dev., 2(1):3-15, 1992.
Kaur et al., Biochemistry, 45(23):7347-55, 2006.
Ketting et al., Cell, 99(2):133-141, 1999.
Korn et al., Glia, 49(1):73-83, 2005.
Koshkin et al., Bioorg. Med. Chem. Lett., 8(16):2219-22, 1998.
Krützfeldt et al., Nature, 438(7068):685-9, 2005.
Kumar et al., Biochem. Pharmacol., 55:775-783, 1998.
Larsen and Nielsen, Nucl. Acids Res., 24:458-463, 1996.
Lin et al., Genetics, 153:1245-1256, 1999.
Maurer et al., Mol Membr Biol., 16(1):129-40, 1999.
McGarry and Lindquist, Proc. Natl. Acad. Sci. USA, 83(2):399-403, 1986.
McMahon et al., Life Sci., 71(3):325-3, 2002.
Mollegaard et al., Proc. Natl. Acad. Sci. USA, 91:3892-3895, 1994.
Montgomery et al., Proc. Natl. Acad. Sci. USA, 95:15502-15507, 1998.
Morita et al., Bioorg. Med. Chem., 11(10):2211-26, 2003.
Morris et al., Science, 305:1289-1292, 2004.
Nielsen et al., Science, 254:1497-1500, 1991.
Nielsen, In: Polyamide nucleic acids, in Antisense Research and Applications, Crooke and Lebleu (Eds.), CRC Press, Boca Raton, Fla., Chap. 20, 1993.
Nucleic Acids in Chemistry and Biology, Blackburn and Gait (Eds.), IRL Press at Oxford University Press, Inc. NY, 1990.
Ohkawa et al., Nucleic Acids Symp Ser., (27):15-6, 1992
Ojwang et al., Proc. Natl. Acad. Sci. USA, 89(22):10802-6, 1992.
Oligonucleotides and Analogues: A Practical Approach, Eckstein (Ed.), IRL Press at Oxford University Press, Inc. NY, 1991
Ozakbas et al., J. Neural. Sci., 218(1-2):3-7, 2004.
Paroo and Corey, Trends Biotechnol., 22(8):390-394, 2004.
PCT Appln. WO98/24893
PCT Appln. WO 00/44914
PCT Appln. WO 00/53722
PCT Appln. WO 01/68836
PCT Appln. WO 05/021570
PCT Appln. WO 05/121371
PCT Appln. WO 05/115481
PCT Appln. WO 93/23569
PCT Appln. WO 94/02595
PCT Appln. WO 94/02595
PCT Appln. WO 94/14226
PCT Appln. WO 98/39352
PCT Appln. WO 99/14226
PCT Appln. WO 99/14226
PCT Appln. WO 99/32619
PCT Appln. WO 01/36646
Prakash et al., J. Med. Chem., 48:4247-4253, 2005.
Rand et al., Cell, 123:621-629, 2005.
Remington's Pharmaceutical Sciences, 18th edition, Gennaro (Ed.), Mack Publishing Co., Easton, Pa., 1990.
Renno et al., J. Immunology, 168:4946-4950, 2002.
Riechmann et al., Nature, 332:323-327, 1988.
Sarver et al., Science, 247:1222-1225, 1990.
Scanlon et al., Proc. Natl. Acad. Sci. USA, 88:10591-10595, 1991.
Sharp et al., J. Cereb. Blood Flow Metab., 20:1011-1032, 2000.
Sharp, Genes Dev., 13:139-141, 1999.
Sims et al., J. Immunol., 151:2296, 1993.
Singh et al., J. Biol. Chem., 273:20354-20362, 1998.
Soutschek et al., Nature, 432(7014):173-178, 2004.
Summerton and Weller, Antisense Nucleic Acid Drug Dev., 7(3):187-95, 1997.
Tabara et al., Cell, 99(2):123-132, 1999.
Taira et al., Nucleic Acids Res., 19(19):5125-30, 1991.
Taylor et al., Oncogene, 21:5765-5772, 2002.
Thompson et al., Nature Genet., 9:444-450, 1995.
Thompson et al., Nature Medicine, 1:277-278, 1995.
Toole, Curr. Top. Dev. Biol., 54:371-389, 2003.
Tyler et al. Proc. Natl. Acad. Sci. USA, 8;96(12):7053-8, 1999b.
Tyler et al., Am. J. Physiol., 277(6 Pt 1):L1199-204, 1999a.
Urban-Klein, Gene Ther., 12(5):461-6, 2005.
Urn et al., FEBS Lett., 441:88-92, 1998.
Vaughan et al., Nature Biotechnol., 16:535-539, 1998.
Ventura et al., Nucleic Acids Res., 21(14):3249-55, 1993.
Verhoeyen et al., Science, 239:1534-1536, 1988.
Wahlestedt et al., Proc. Natl. Acad. Sci. USA, 97(10):5633-5638, 2000.
Weerasinghe et al., J. Virol., 65(10):5531-5534, 1991.
Wincott et al., Nucleic Acids Res., 23(14):2677-2684, 1995.
Yong et al., Nature Rev. Neurosci., 2:502-511, 2001.
Zhang et al., Eur. J. Nucl. Med., 11:1700-1707, 2000.

1. A method of inhibiting entry of leukocytes into the central nervous system (CNS) of a subject comprising administering to the subject an effective amount of a first molecule that inhibits the activity of EMMPRIN (CD147).

2. The method of claim 1, wherein the molecule is an antibody or antibody fragment that binds immunologically to EMMPRIN.

3. The method of claim 2, wherein said antibody is a scFv, scFab, Fab, chimeric or humanized antibody.

4. The method of claim 1, wherein inhibiting activity comprises inhibiting EMMPRIN expression.

5. The method of claim 4, wherein the molecule is an siRNA or miRNA that inhibits EMMPRIN expression.

6. The method of claim 5, wherein the siRNA targets an exon or an intron/exon junction.

7. The method of claim 5, wherein the miRNA targets an exon or an intron.

8. The method of claim 1, wherein the administering is intravenous or intraperitoneal.

9. The method of claim 1, wherein the administering is oral or via inhalation.

10. The method of claim 1, wherein said subject suffers from multiple sclerosis (MS), and said molecule reduces one or more symptoms of multiple sclerosis.

11. The method of claim 1, wherein said subject suffers from multiple sclerosis (MS), and said molecule delays the progression of one or more symptoms of multiple sclerosis.

12. The method of claim 1, wherein said subject is at risk of developing or has subclinical multiple sclerosis (MS), and said molecule delays the onset of one or more multiple sclerosis symptoms.

13. The method of claim 10, further comprising administering to said subject a second anti-MS therapy.

14. The method of claim 13, wherein said second anti-MS therapy may comprise Avonex®, CinnoVex®, ReciGen®, Rebif®, Betaseron®, Copaxone®, Novantrone®, or Tysabri®.

15. The method of claim 1, further comprising at least a second administering of said molecule.

16. The method of claim 1, further comprising chronic administering of said molecule.

17. The method of claim 1, further comprising administering a second molecule that inhibits the activity of EMMPRIN.

18. The method of claim 17, wherein said first molecule is an anti-EMMPRIN antibody that prevents leukocyte cell adhesion, and said second molecule is an anti-EMMPRIN antibody that inhibits leukocyte cell proliferation.

19. The method of claim 17, wherein said first molecule is an anti-EMMPRIN antibody, and said second molecule is an EMMPRIN siRNA.

20. The method of claim 17, wherein said first molecule is a first EMMPRIN siRNA, and said second molecule is a second EMMPRIN siRNA.


SEQUENCE LISTING (4 sequences)

SEQ ID NO: 1 - protein, 44 aa, Mus musculus
TIQTSVQEVNSKTQLTCSLNSSGVDIVGHRWMRGGKVLQEDTLP

SEQ ID NO: 2 - protein, 43 aa, Homo sapiens
TVFTTVEDLGSKILLTCSLNDSATEVTGHRWLKGGVVLKEDAL

SEQ ID NO: 3 - protein, 16 aa, Artificial Sequence
  Synthetic peptide
TCSLNDSATEVTGHRW

SEQ ID NO: 4 - protein, 17 aa, Artificial Sequence
  Synthetic peptide
GHRWLKGGVVLKEDALP